(19) United States
(12) Patent Application Publication
(10) Pub. No.: US 2022/0010321 A1
(43) Pub. Date: Jan. 13, 2022

(54) DUAL GUIDE RNA FOR CRISPR/CAS GENOME EDITING IN PLANTS CELLS

(71) Applicant: KEYGENE N.V., Wageningen (NL)

(72) Inventor: Franck Georges Paul LHUISSIER, Wageningen (NL)

(73) Assignee: KEYGENE N.V., Wageningen (NL)

(21) Appl. No.: 17/290,547

(22) PCT Filed: Nov. 1, 2019
(86) PCT No.: PCT/EP2019/079950

§ 371 Date: Apr. 30, 2021

(30) Foreign Application Priority Data
Nov. 1, 2018 (EP) 18203919.8

(51) Int. Cl. C12N 15/82; C12N 15/11; C12N 9/22

(52) U.S. Cl. CPC C12N 15/8213; C12N 2310/20; C12N 9/22; C12N 15/111

(57) ABSTRACT

The invention pertains to a method for targeted modification of DNA in a plant cell, comprising a step of contacting the DNA with an RNA-guided CRISPR-system nuclease complex, wherein the complex comprises a crRNA and a tracrRNA as separate molecules. The invention further pertains to said RNA-guided CRISPR-system nuclease complex for targeting of DNA in a plant cell and kits comprising the RNA-guided CRISPR-system nuclease complex or constructs encoding the same.

### FIELD OF THE INVENTION

The present invention is in the field of molecular biology and plant biology. The invention concerns targeted DNA modifications, including methods and compositions for making such modifications.

### BACKGROUND

The process of deliberately creating changes in the genetic material of living cells has the goal of modifying one or more genetically encoded biological properties of that cell, or of the organism of which the cell forms part or into which it can regenerate. These changes can e.g. take the form of deletion of parts of the genetic material, addition of exogenous genetic material, or changes in the existing nucleotide sequence of the genetic material. Methods of altering the genetic material of eukaryotic organisms have been known for over 20 years, and have found widespread application in plant, human and animal cells and micro-organisms for improvements in the fields of agriculture, human health, food quality and environmental protection. The most common methods consist of adding exogenous DNA fragments to the genome of a cell, which will then confer a new property to that cell or its organism over and above the properties encoded by already existing genes, including applications in which the expression of existing genes will thereby be suppressed. Although many such examples are effective in obtaining the desired properties, these methods have several drawbacks. For example, these conventional methods are not very precise, because there is not always control over the genomic positions in which the exogenous DNA fragments are inserted (and hence over the ultimate levels of expression), and the desired effect will have to manifest itself over the natural properties encoded by the original and well-balanced genome. On the contrary, methods of genome editing that will result in the addition, deletion or conversion of nucleotides in predefined genomic loci will allow the precise modification of existing genes.

By using site-specific nucleases, such as zinc finger nucleases (ZFNs), transcription activator like effector nucleases (TALENs), and Clustered Regularly Interspaced Short Palindromic Repeat (CRISPR) nucleases, the field of targeted DNA alteration is rapidly developing.

CRISPRs (Clustered Regularly Interspaced Short Palindromic Repeats) are loci containing multiple short direct repeats and are found in 40% of the sequenced bacteria and 90% of sequenced archaea. The CRISPR repeats form a system of acquired bacterial immunity against genetic pathogens such as bacteriophages and plasmids. When a bacterium is challenged with a pathogen, a small piece of the pathogen's genome is processed by CRISPR associated proteins (Cas) and incorporated into the bacterial genome between CRISPR repeats. The CRISPR loci are then transcribed and processed to form so called crRNAs which include approximately 30 bps of sequence identical to the pathogen's genome. These RNA molecules form the basis for the recognition of the pathogen upon a subsequent infection and lead to silencing of the pathogen genetic elements through direct digestion of the pathogen's genome. The Cas9 protein is an essential component of the type-II CRISPR/Cas system from S. pyogenes and forms an endonuclease, when combined with the crRNA and a second RNA termed the trans-activating crRNA (tracrRNA), which targets the invading pathogenic DNA for degradation by the introduction of DNA double strand breaks (DSBs) at the position in the genome defined by the crRNA. This type-II CRISPR/Cas9 system has been proven to be a convenient and effective tool in biochemistry that, via the targeted introduction of double-strand breaks and the subsequent activation of endogenous repair mechanisms, is capable of introducing modification in eukaryotic genomes at sites of interest. Jinek et al. (2012, Science 337: 816-820) demonstrated that a single chain chimeric RNA (single guide RNA, sRNA, sgRNA), produced by combining the essential sequences of the crRNA and tracrRNA into a single RNA molecule, was able to form a functional endonuclease in combination with Cas9. Many different CRISPR/Cas systems have been identified from different bacterial species (Zetsche et al. 2015 Cell 163, 759-771; Kim et al. 2017, Nat. Commun. 8, 1-7; Ran et al. 2015. Nature 520, 186-191).

The CRISPR/Cas9 system can be used for genome editing in a wide range of different organisms and cell types. First a genomic sequence is identified at which the CRISPR/Cas endonuclease should induce a DSB and this is then screened for the presence of a protospacer adjacent motif (PAM). The PAM sequence is essential for the CRISPR/Cas endonuclease activity, is relatively short, and is therefore usually present multiple times in any given sequence of some length. For instance the PAM motif of the S. pyogenes Cas9 protein is NGG, which ensures that for any given genomic sequence multiple PAM motifs are present and so many different guide RNAs can be designed. In addition, guide RNAs can also be designed targeting the opposite strands of the same double strand sequence. The sequence immediately adjacent to the PAM is incorporated into the guide RNA. This can differ in length depending upon the CRISPR/Cas system being used. For instance, the optimal length for the targeting sequence in the Cas9 sgRNA is 20nt, and in most cases a sequence of this length is unique in a plant genome. For expression in plant cells a gene coding for a guide RNA can be linked to an RNA polymerase-III promoter, such as the U6 promoter from Arabidopsis, or the corresponding or functionally similar pol-III promoter from the cell type, organism, plant species or family in which the experiments are being performed.

The CRISPR/Cas endonuclease can be expressed in the cell from any form of constitutive or inducible promoter that is suitable for the organism or cell type in which the experiments are being performed. In some instances, the protein expression levels of the CRISPR/Cas endonuclease can be improved by optimization of its codon usage for the specific cell type or organism.

The two components of the CRISPR/Cas system, the endonuclease and the guide RNA(s) can be expressed in the cell from ectopic genomic elements such as (non-replicating) plasmid constructs, viral vectors or introduced directly in the cells or organism as protein (the CRISPR/Cas endonuclease) and RNA (guide RNA). In addition mRNA encoding the CRISPR/Cas endonuclease can be used. When the plasmid or viral vectors are unable to replicate in the transformed cells then the CRISPR/Cas and guide RNA(s) are expressed or present for a short period and then are eliminated from the cell. Stable expression of the CRISPR/Cas protein and guide RNA can be achieved using a transgenic approach whereby the genes coding for them are integrated into the host genome.

Once the CRISPR/Cas endonuclease and the guide RNA is present/expressed in the cell then the complex of the two components scans the genomic DNA for the sequence complementary to the targeting sequence on the guide RNA and adjacent to a PAM sequence. Depending on the CRISPR/Cas endonuclease being used, the complex then induces nicks in both of the DNA strands at varying distances from the PAM. For instance the S. pyogenes Cas9 protein introduces nicks in the both DNA strands 3 bps upstream from the PAM sequence to create a blunt DNA DSB.

Once a DNA DSB has been produced the cellular DNA repair machinery, particularly proteins belonging to the non-homologous end joining (NHEJ) pathway, are involved in the re-ligation of the DNA ends. If this DSB is repaired accurately then the sequence again forms a target for cutting by the CRISPR/Cas-guide RNA complex. However, some re-ligation events are imprecise and can lead to the random loss or gain of a few nucleotides at the break, resulting in an indel mutation in the genomic DNA. This results in an alteration of the target sequence that prevents binding of the guide RNA and thus any further DSB induction.

The generated indel may e.g. disrupt a transcription factor binding site or a splice site. In case the indel is generated in the coding sequence, the open reading frame may be altered resulting in a null mutation.

Alternatively, the DNA DSB may be repaired in the presence of an introduced single-stranded or double-stranded DNA template. Using such template, one or more nucleotides can be specifically introduced and/or altered in the DNA sequence.

The CRISPR/Cas9 system, comprising Cas9, modified by comprising a nuclear localization signal, complexed with single chain chimeric RNA (sgRNA) as originally designed by Jinek et al (Jinek et al. (2012, Science 337: 816-820) has till now been used successfully in genome editing of plant cells. However using such sgRNA, the plant genome editing efficiency is still relatively low. There is therefore a need in the art for a more efficient targeted modification of plant cells. In addition, there is a need in the art for a more efficient targeted modification of plant cells, whereby specific nucleotides are altered and/or introduced.

### SUMMARY

In a first aspect, the invention pertains to a method for targeted modification of DNA in a plant cell, comprising a step of contacting the DNA with an RNA-guided CRISPR-system nuclease complex, wherein said complex comprises a CRISPR-system nuclease, a crRNA and a tracrRNA and wherein the crRNA and the tracrRNA are separate (non-covalently linked) molecules.

Preferably, the CRISPR-system nuclease comprises two catalytically active endonuclease domains.

Preferably, the CRISPR-system nuclease comprises at least one catalytically inactive endonuclease domain.

In an embodiment, the CRISPR-system nuclease is fused to a functional domain, preferably, a deaminase domain.

Preferably, the CRISPR-system nuclease is introduced in the cell by transfecting the cell with a vector encoding said CRISPR-system nuclease.

Preferably, the CRISPR-system nuclease is introduced in the cell by transfecting the cell with the CRISPR-system nuclease.

In an embodiment, at least one of the crRNA and tracrRNA is introduced in the cell by transfecting the cell with a vector encoding said crRNA and/or tracrRNA.

In an embodiment, at least one of the crRNA and tracrRNA is introduced in the cell by transfecting the cell with said crRNA and/or tracrRNA, and wherein preferably the crRNA and/or tracrRNA is chemically modified.

Preferably, the cell is further transfected with a template oligonucleotide, wherein preferably the template oligonucleotide is chemically modified.

Preferably, the cell is further transfected with a donor construct, wherein preferably the donor construct is chemically modified.

In an embodiment, the CRISPR-system endonuclease, crRNA, tracrRNA and/or optionally the template oligonucleotide or donor construct, are introduced into the plant cell using polyethylene glycol mediated transfection, preferably using an aqueous medium comprising PEG.

Preferably, the method further comprises the step of regenerating a plant or descendent thereof comprising the targeted modification.

In an aspect, the invention further pertains to an RNA-guided CRISPR-system nuclease complex comprising the CRISPR-system nuclease, the crRNA and the tracrRNA as defined herein, or one or more constructs encoding the same, for targeted modification of DNA in a plant cell.
In a further aspect, the invention relates to a kit for targeted modification of DNA in a plant cell comprising at least one of
   i) a container comprising the CRISPR-system nuclease as defined herein; and
   ii) a container comprising one or more constructs encoding a CRISPR-system nuclease as defined herein,

and optionally a container comprising a tracrRNA and/or one or more crRNAs, and/or constructs encoding the same.

In an aspect, the invention concerns the use of a RNA guided CRISPR-system nuclease complex as defined herein, or one or more constructs encoding the same, or a kit as defined herein, for targeted modification of DNA in a plant cell.

### FIGURE LEGEND

FIG. 1. Comparison of sgRNA and cr/tracr RNA. Shown is the percentage of indels (black bars), clean gene targeting (white bars) and SNP introduction (fluorescence, hatched bars) in relation to the total number of target sequence reads or total number of transfected protoplasts, found after transfection of Arabidopsis protoplasts with ribonucleoprotein complexes comprising either a sgRNA or a combination of a crRNA and a tracrRNA (cr/tracrRNA), in absence (−) or presence (+) of a single-stranded oligonucleotide (ssODN).

### DEFINITIONS

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N.Y., 1989; Ausubel et al., Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. The indefinite article “a” or “an” thus usually means “at least one”. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: The term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

Exemplary”: this terms means “serving as an example, instance, or illustration,” and should not be construed as excluding other configurations disclosed herein.

“Plant”: Refers to either the whole plant or to parts of a plant, such as cells, tissue cultures or organs (e.g. pollen, seeds, ovules, gametes, roots, leaves, flowers, flower buds, branches, anthers, fruit, kernels, ears, cobs, husks, stalks, root tips, grains, embryos, etc.) obtainable from the plant, as well as derivatives of any of these and progeny derived from such a plant by selling or crossing. “Plant” further includes plant protoplasts, plant cell tissue cultures from which plants can be regenerated, plant calli, plant clumps, and plant cells that are intact in plants or parts of plants such as embryos, pollen, ovules, gametes, seeds, leaves, flowers, branches, fruit, kernels, ears, cobs, husks, stalks, roots, root tips, anthers, grains and the like. “Plant cell(s)” include protoplasts, gametes, suspension cultures, microspores, pollen grains, etc., either in isolation or within a tissue, organ or organism.

The terms “construct”, “nucleic acid construct”, “vector”, and “expression vector” are used interchangeably herein and is herein defined as a man-made nucleic acid molecule resulting from the use of recombinant DNA technology. These constructs and vectors therefore do not consist of naturally occurring nucleic acid molecules although a vector may comprise (parts of) naturally occurring nucleic acid molecules. A vector can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g. a coding sequence, an antisense or an inverted repeat sequence) is integrated downstream of the transcription regulatory sequence. Vectors may comprise further genetic elements to facilitate their use in molecular cloning, such as e.g. selectable markers, multiple cloning sites and the like. The vector backbone may for example be a binary or superbinary vector (see e.g. U.S. Pat. No. 5,591,616, US 2002138879 and WO 95/06722), a co-integrate vector or a T-DNA vector, as known in the art.

Expression vectors according to the invention are particularly suitable for introducing gene expression in a cell, preferably a plant cell. A preferred expression vector is a naked DNA, a DNA complex or a viral vector, wherein the DNA molecule can be a plasmid. A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, a dendrimer, an inorganic nanoparticle, a virosome and cell-penetrating peptides. In a preferred embodiment the expression vector is a viral vector, preferably a Tobacco Rattle Virus (TRV), a Bean yellow dwarf virus (BeYDV), a Cabbage leaf curl virus (CaLCuV), a tobravirus and a Wheat dwarf virus (WDV). Preferably, the viral vector is a Tobacco Rattle Virus as defined herein above

The term “gene” means a DNA fragment comprising a region (transcribed region), which is transcribed into an RNA molecule (e.g. a pre-mRNA or ncRNA) in a cell. The transcribed region can be operably linked to suitable regulatory regions (e.g. a promoter), which form part of the gene as defined herein. A gene can comprise several operably linked fragments, such as a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, and, in case the RNA encodes for a biologically active protein or peptide, subsequently translated into a biologically active protein or peptide.

The term “operably linked” refers to a linkage of polynucleotide elements in a functional relationship. A nucleic acid is “operably linked” when it is placed into a functional relationship with another nucleotide sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation). A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

The terms “protein” or “polypeptide” are used interchangeably herein and refer to molecules consisting of a chain of amino acids, without reference to a specific mode of action, size, 3 dimensional structure or origin. A “fragment” or “portion” of a protein may thus still be referred to as a “protein.” A protein as defined herein and as used in any method as defined herein may be an isolated protein. An “isolated protein” is used to refer to a protein which is no longer in its natural environment, for example in vitro or in a recombinant bacterial or plant host cell.

The term “regeneration” is herein defined as the formation of a new tissue and/or a new organ from a single plant cell, a callus, an explant, a tissue or an organ. Preferably, the regeneration is at least one of shoot regeneration, ectopic apical meristem formation, and root regeneration. Regeneration can occur through somatic embryogenesis or organogenesis. The regeneration may further include the formation of a new plant from a single plant cell or from e.g. a callus, an explant, a tissue or an organ. The plant cell for regeneration can be an undifferentiated plant cell. The regeneration process hence can occur directly from parental tissues or indirectly, e.g. via the formation of a callus.

“Conditions that allow for regeneration” is herein understood as an environment wherein a plant cell or a tissue can regenerate. Such conditions include at minimum a suitable temperature, nutrition, day/night rhythm and irrigation.

The term “deaminase” refers to an enzyme that catalyzes a deamination reaction. In some embodiments, the deaminase is a cytosine deaminase, catalyzing the hydrolytic deamination of cytosine to uracil. The deaminase may also be an adenine deaminase, catalyzing the deamination of adenine thereby converting it to inosine.

“Nucleotide sequence”: This refers to the order of nucleotides of, or within a nucleic acid. In other words, any order of nucleotides in a nucleic acid may be referred to as a sequence or nucleotide sequence.

“Amino acid sequence”: This refers to the order of amino acid residues of, or within a protein. In other words, any order of amino acids in a protein may be referred to as amino acid sequence.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “homolog” of a gene is a further gene by descent from a common ancestral DNA sequence. The term homolog may apply to the relationship between genes separated by the event of speciation (ortholog) or to the relationship between genes separated by the event of genetic duplication (paralog).

An “ortholog” of a gene is a gene in a different species that evolved from a common ancestral gene by speciation, and is understood herein as having retained the same function in the course of evolution.

A “target sequence” is to denote an order of nucleotides within a nucleic acid that is to be targeted (e.g. wherein an alteration is to be introduced or to be detected. For example, the target sequence is an order of nucleotides comprised by a first strand of a DNA duplex.

The “protospacer sequence” is the sequence that is located in, at or near the target sequence that is hybridisable or targeted by a crRNA.

An “endonuclease” is an enzyme that hydrolyses at least one strand of a duplex DNA upon binding to its recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

### DETAILED DESCRIPTION

The wild-type CRISPR-system may use a crRNA and a separate tracrRNA to bind to and cleave specific locations of a DNA molecule in archaea and bacteria. In the past, these separate molecules have been covalently linked via a linker molecule to form a single molecule, i.e. a single guide RNA (sgRNA, see e.g. WO2013/176772) which is now commonly used for genome editing.

The inventors discovered that a CRISPR/Cas system based on a guide RNA comprising a crRNA and tracrRNA as separate (non-covalently linked) molecules is surprisingly more effective in plant cells as compared to the same system making use of a single guide (sg)RNA. In particular the inventors discovered that in comparison to the conventional single-guide RNA, the combination of a crRNA and tracrRNA as separate molecules, which together hybridize to form a guide RNA, also named herein a dual guide RNA, increased the number of generated indels. In addition in the presence of a single-stranded oligonucleotide, targeted nucleotide exchange was significantly more efficient when using a dual guide RNA, instead of a single-guide RNA.

Hence, in a first aspect the invention pertains to a method for targeted modification of DNA in a plant cell, comprising a step of contacting the DNA with an RNA-guided CRISPR-system nuclease complex, wherein said complex comprises the following components:

i) a CRISPR-system nuclease;

ii) a crRNA; and

iii) a tracrRNA.

The crRNA and the tracrRNA within the complex are separate molecules that are capable of interacting, i.e. by non-covalent binding (e.g. through hybridization), to form a dual guide RNA. Therefore in an embodiment, the 3′ end of the crRNA, is not covalently-linked to the 5′ end of the tracrRNA. Preferably, the tracrRNA and crRNA may form a two-RNA structure, i.e. a dual guide RNA, that directs the CRISPR-system nuclease to a specific location in the DNA.

In an embodiment of the invention, the method comprises a step of contacting the DNA with a complex as defined herein.

The DNA may be any type of DNA, endogenous or exogenous to the plant cell, for example genomic DNA, chromosomal DNA, artificial chromosomes, plasmid DNA, or episomal DNA. The DNA may be nuclear or organellar DNA. Preferably, the DNA is chromosomal DNA. The chromosomal DNA can be in vitro or in vivo, preferably in vivo. Preferably the chromosomal DNA is endogenous to the plant cell.

The method of the invention results in altered DNA at a site of interest. The site of interest preferably comprises or consists of a target sequence. The target sequence can be, or can be part of, any site of interest in the DNA. Preferably, the DNA target sequence is flanked by or comprises a PAM sequence known for interacting with the CRISPR-system nuclease of the complex as defined herein (e.g. see Ran et al 2015, Nature 520:186-191). For instance, if said CRISPR-nuclease is S. pyogenes Cas9, the PAM sequence may have a sequence of 5′-NGG-3′. For instance, for Geobacillus thermodenitrificans T12 Cas9 (e.g. see WO2016/198361) the PAM sequence may have a sequence of 5′-NNNNCNNA-3′ (SEQ ID NO: 1). Further known PAM sequences for Cas9 endonucleases are: Type IIA 5′-NGGNNNN-3′ (Streptococcus pyogenes), 5′-NNGTNNN-3′ (Streptococcus pasteurianus), 5′-NNGGAAN-3′ (Streptococcus thermophilus), 5′-NNGGGNN-3′ (Staphylococcus aureus), and Type IIC 5′-NGGNNNN-3′ (Corynebacterium difteriae), 5′-NNGGGTN-3′ (Campylobacter lari), 5′-NNNCATN-3′ (Parvobaculum lavamentivorans) and 5′-NNNNGTA-3′ (Neiseria cinerea).

Preferably, the PAM site is recognized by the CRISPR-system nuclease.

The target sequence can be, or can be complementary to, a coding sequence or a non-coding sequence. The non-coding sequence can be within a gene, such as, but not limited to, at least one of a regulatory sequence, an intronic sequence and a sequence comprising a splice site. Alternatively, the target sequence can be, or can be complementary to, an intergenic sequence or a sequence encoding a non-coding RNA molecule which may be a regulatory RNA molecule. The target sequence can be a coding sequence, e.g. a sequence encoding a protein.

The site of interest can be present only once, i.e. is unique, in the DNA. Alternatively, the site of interest can be present at least 1, 2, 3, 4, 5, 6, 7, 8, 9 or at least 10 times in the DNA.

The site of interest is preferably within a gene of interest, preferably within a gene at the plants endogenous chromosomal DNA. In other words, the method of the invention preferably results in a genomic modification, which may refer to an epigenetic modification and/or a genetic modification. The terms “alteration” and “modification” are used interchangeably herein. An epigenetic modification is a heritable modification that changes the gene function or activity, without changing the nucleotide sequence. In an embodiment, the modification is a genetic modification. A genetic modification is understood herein as the alteration of the nucleotide sequence of the DNA, such as a deletion, insertion, substitution or conversion of one or more nucleotides.

The method of the invention may further comprise a step of multiplication and/or genotyping using any conventional method known in the art, in order to screen or test for the nucleotide alteration. The method of the invention therefore comprises such step after contacting the DNA with the RNA-guided CRISPR-system nuclease complex. In a preferred embodiment, the cells, preferably the plant cells, comprising the targeted modification as defined herein, may be genotyped using deep-sequencing technologies, such as Illumina or 454 sequencing.

The targeted DNA modification may be within a coding sequence in the plant genome, thereby resulting in a modified protein, e.g. a protein comprising one or more amino acid alterations or a protein comprising a truncation. The targeted DNA modification may be within a noncoding sequence in the plant genome, such as in an intronic sequence or in a sequence encoding a noncoding (nc)RNA. The targeted modification in a noncoding sequence may result in e.g. a modified splice site or an alteration in the regulatory function of a non-coding RNA. The targeted nucleotide alteration may also be in a regulatory sequence resulting in the down or upregulation of gene expression, optionally in knocking out gene expression.

The method of the invention may comprise a step of screening or testing for protein modifications and/or protein expression levels. Such screening or testing may be directly on the protein itself or on altered functionality using any conventional means. In addition or alternatively, the DNA modification may result in a phenotypic alteration of the plant cell or plant. Therefore, the method may comprise a step of screening or testing for a phenotypic alteration or characteristic in the plant cell or plant, preferably a step of screening or tested for a phenotypic characteristic as defined herein.

Preferably, the method of the invention results in a genetic modification of a gene of interest.

### Gene of Interest

Preferably, a gene of interest (GOI) is a gene that produces or alters a characteristic, preferably a phenotypic characteristic. A plant GOI thus preferably produces or alters a plant characteristic, preferably a phenotypic plant characteristic. The term “plant characteristic” means any characteristic of a plant, plant cell or plant tissue. Preferably, the plant characteristic is selected from the group consisting of plant development, plant growth, yield, biomass production, plant architecture, plant biochemistry, plant physiology, metabolism, survival capacity and stress tolerance. Alternatively or in addition, the plant characteristic is selected from the group consisting of DNA synthesis, DNA modification, endoreduplication, cell cycle, cell wall biogenesis, transcription regulation, signal transduction, storage lipid mobilization, and photosynthesis.

The term “altering a plant characteristic” as used herein encompasses any change in the plant characteristic such as increase, decrease or change in time or place. It is understood herein that the plant GOI can alter the plant characteristic by introducing, increasing, decreasing, or removing the expression of the GOI and/or by modifying the functionality of the encoded protein such as by altering the coding sequence thereby resulting in expression of a modified encoded protein. Whether the plant characteristic is altered due to an introduced expression of the GOI, increased expression of the GOI, decreased expression of the GOI, removed expression of the GOI and/or modified functionality of the encoded protein, is dependent on the type of GOI and/or the type of plant characteristic.

In an embodiment, the targeted modification is genetic modification that alters a plant characteristic. Such modification may be an early stop. Such modification may also be a single nucleotide modification (SNP) resulting in an amino acid change in the translated protein, which may result in a single amino acid change.

Detailed herein below are, non-limiting, examples of plant characteristics that can be modified by the method of the invention as described herein:

“Growth” refers to the capacity of the plant or of plant parts to expand and increase in biomass. Altered growth refers amongst others to altered growth rate, cycling time, the size, expansion or increase of the plant. Additionally and/or alternatively, growth characteristics may refer to cellular processes comprising, but not limited to, cell cycle (entry, progression, exit), cell division, cell wall biogenesis and/or DNA synthesis, DNA modification and/or endoreduplication.

“Yield” refers to the harvestable part of the plant. “Biomass” refers to any part of the plants. These terms also encompass an increase in seed yield, which includes an increase in the biomass of the seed (seed weight) and/or an increase in the number of (filled) seeds and/or in the size of the seeds and/or an increase in seed volume, each relative to corresponding wildtype plants. An increase in seed size and/or volume may also influence the composition of seeds. An increase in seed yield could be due to an increase in the number and/or size of flowers. An increase in yield may also increase the harvest index, which is expressed as a ratio of the total biomass over the yield of harvestable parts, such as seeds.

“Plant development” means any cellular process of a plant that is involved in determining the developmental fate of a plant cell, in particular the specific tissue or organ type into which a progenitor cell will develop. Typical plant characteristics according to the present invention are therefore characteristics relating to cellular processes relevant to plant development such as for example, morphogenesis, photomorphogenesis, shoot development, root development, vegetative development, reproductive development, stem elongation, flowering, regulatory mechanisms involved in determining cell fate, pattern formation, differentiation, senescence, time of flowering and/or time to flower.

Plant architecture”, as used herein refers to the external appearance of a plant, including any one or more structural features or a combination of structural features thereof. Such structural features include the shape, size, number, position, colour, texture, arrangement, and patternation of any cell, tissue or organ or groups of cells, tissues or organs of a plant, including the root, stem, leaf, shoot, petiole, trichome, flower, petal, stigma, style, stamen, pollen, ovule, seed, embryo, endosperm, seed coat, aleurone, fibre, fruit, cambium, wood, heartwood, parenchyma, aerenchyma, sieve element, phloem or vascular tissue, amongst others.

The term “stress tolerance” is understood as the capability of better survival and/or better performing in stress conditions such as environmental stress, which can be biotic or abiotic. Salinity, drought, heat, chilling and freezing are all described as examples of conditions which induce osmotic stress. The term “environmental stress” as used in the present invention refers to any adverse effect on metabolism, growth or viability of the cell, tissue, seed, organ or whole plant which is produced by a non-living or non-biological environmental stressor. More particularly, it can encompass environmental factors such as water stress (flooding, water logging, drought, dehydration), anaerobic (low level of oxygen, CO2 etc.), aerobic stress, osmotic stress, salt stress, temperature stress (hot/heat, cold, freezing, frost) or nutrients deprivation, pollutants stress (heavy metals, toxic chemicals), ozone, high light, pathogen (including viruses, bacteria, fungi, insects and nematodes) and combinations of these. Biotic stress is stress as a result of the impact of a living organism on the plant. Examples are stresses caused by pathogens (virus, bacteria, nematodes insects etc.). Another example is stress caused by an organism, which is not necessarily harmful to the plant, such as the stress caused by a symbiotic or an epiphyte. Accordingly, particular plant characteristics obtained by modification of the GOI can encompass early vigour, survival rate, stress tolerance.

Characteristics related to “plant physiology” can encompass characteristics of functional processes of a plant, including developmental processes such as growth, expansion and differentiation, sexual development, sexual reproduction, seed set, seed development, grain filling, asexual reproduction, cell division, dormancy, germination, light adaptation, photosynthesis, leaf expansion, fiber production, secondary growth or wood production, amongst others; responses of a plant to externally-applied factors such as metals, chemicals, hormones, growth factors, environment and environmental stress factors (e.g. anoxia, hypoxia, high temperature, low temperature, dehydration, light, day length, flooding, salt, heavy metals, amongst others), including adaptive responses of plants to said externally-applied factors. Particular plant physiology characteristics which are altered by the GOI identified in the method of the invention can further encompass altered storage lipid mobilization, photosynthesis, transcription regulation and signal transduction.

Plant characteristics related to “plant biochemistry” are to be understood by those skilled in the art to preferably refer to the metabolic characteristics. “Metabolism” can be used interchangeable with biochemistry. Metabolism and/or biochemistry encompass catalytic or assimilation or other metabolic processes of a plant, including primary and secondary metabolism and the products thereof, including any element, small molecules, macromolecules or chemical compounds, such as but not limited to starches, sugars, proteins, peptides, enzymes, hormones, growth factors, nucleic acid molecules, celluloses, hemicelluloses, calloses, lectins, fibres, pigments such as anthocyanins, vitamins, minerals, micronutrients, or macronutrients, that are produced by plants.

The modification of the GOI can be identified by determining the altered plant characteristic, preferably by determining at least one or more altered plant characteristics as defined herein above. In an embodiment, the plant cell having the preferred altered plant characteristic is selected and isolated from plant cells not having the altered plant characteristic. The plant cell can first be generated into a plant prior to selecting the plant having the altered plant characteristic.

The plant cell or plant having an altered plant characteristic can be sequenced to identify and/or further analyse the gene of interest. The skilled person understands that the whole plant genome can be sequenced or a part of the plant genome. In an embodiment, at least the modified sequence is determined.

Preferably, in those cases wherein the modification of the GOI results in an altered plant characteristic, the modification identifies a gene of interest. Therefore the method of the invention can be used in order to screen for gene functionality.

tracrRNA and crRNA
The RNA-guided CRISPR-system nuclease complex for use in a method as defined herein comprises, in addition to a CRISPR-system nuclease, a crRNA and a tracrRNA. Molecules suitable as crRNA and tracrRNA are well known in the art (see e.g., WO2013142578 and Jinek et al., Science (2012) 337, 816-821). The crRNA comprises a sequence that can hybridize to or near a target sequence, preferably a DNA target sequence as defined herein. Therefore preferably, the crRNA comprises a nucleotide sequence that is complementary to a sequence in the target DNA, i.e. the protospacer sequence. Preferably, the crRNA is also capable of complexing with the tracrRNA.

The crRNA can comprise or consist of non-modified or naturally occurring nucleotides. Alternatively or in addition, the crRNA can comprise or consist of modified or non-naturally occurring nucleotides, preferably such chemically modified nucleotides are for protecting the crRNA against degradation.

In an embodiment of the invention, the crRNA comprises ribonucleotides and non-ribonucleotides. The crRNA can comprise one or more ribonucleotides and one or more deoxyribonucleotides.

The crRNA may comprise one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-O-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

The crRNA may be chemically modified by incorporation of 2′-O-methyl (M), 2′-O-methyl 3′phosphorothioate (MS), 2′-O-methyl 3′thioPACE (phosphonoacetate) (MSP), or a combination thereof, at one or more terminal nucleotides. Such chemically modified crRNAs can comprise increased stability and/or increased activity as compared to unmodified crRNAs. (Hendel et al, 2015, Nat Biotechnol. 33(9); 985-989). In certain embodiments, a crRNA comprises ribonucleotides in a region that hybridizes to a target DNA. In an embodiment of the invention, deoxyribonucleotides and/or nucleotide analogues can be incorporated in the engineered crRNA structures, such as, without limitation, in the sequence hybridizing to the target DNA, in the sequence interacting with the tracrRNA or in between these sequences.

Alternatively or in addition, the chemically modified nucleotides can be located 5′ and/or 3′ of the sequence hybridizing to the target DNA. The chemically modified sequences can further be located 5′ and/or 3′ of the sequence interacting with the tracrRNA.

In an embodiment, the length of the crRNA can be at least about 15, 20, 25, 30, 32, 34, 36, 38, 40, 42, 44, 46, 48, 50, 52, 54, 56, 58, 60, 65, 70, 75, 80, 85, 90, 95, 100 or more nucleotides in length. In some embodiments, the crRNA is less than about 75, 50, 45, 40, 35, 30, 25 or about 20 nucleotides in length Preferably, the length of the crRNA is about 20-100, 25-80, 30-60 or about 35-50 nucleotides in length.

The part of the crRNA sequence that is complementary to the target DNA sequence is designed to have sufficient complementarity with the target sequence to hybridize with the target sequence and direct sequence-specific binding of a complexed nuclease to the target sequence. Said target sequence preferably is within a GOI as defined herein. The protospacer sequence is preferably adjacent to a protospacer adjacent motif (PAM) sequence, which PAM sequence may interact with the CRISPR nuclease of the RNA-guided CRISPR-system nuclease complex as defined herein. For instance, in case the CRISPR nuclease is S. pyogenes Cas9, the PAM sequence preferably is 5′-NGG-3′, wherein N can be any one of T, G, A or C. The skilled person is capable of engineering the crRNA to target any desired target sequence, preferably by engineering the sequence to be at least partly complementary to any desired target sequence, in order to hybridize thereto. Preferably, the complementarity between part of a crRNA sequence and its corresponding target sequence, when optimally aligned using a suitable alignment algorithm, is at least about 70%, 80%, 85%, 90%, 91%, 92%, 93%, 94%, 95%, 96%, 97%, 98% or 100%. The part of the crRNA sequence that is complementary to the DNA target sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a sequence complementary to the DNA target sequence is less than about 75, 50, 45, 40, 35, 30, 25, 20 nucleotides in length. Preferably, the length of the sequence complementary to the target DNA sequence is at least 17 nucleotides. Preferably the complementary crRNA sequence is about 10-30 nucleotides in length, about 17-nucleotides in length or about 15-21 nucleotides in length. Preferably the part of the crRNA that is complementary to the target DNA is 15, 16, 17, 18, 19, 20, 21, 22, 23, 24 or 25 nucleotides in length, preferably 21 nucleotides.

The part of the crRNA that interacts with the tracrRNA is designed to be sufficiently complementary to the tracrRNA to hybridize to the tracrRNA, and direct the complexed nuclease to the target sequence. Preferably, the complementarity between this part of a crRNA sequence and its corresponding part in the tracrRNA, when optimally aligned using a suitable alignment algorithm, is at least about 50%, 60%, 70%, 80%, 85%, 90%, 91%, 92%, 93%, 94%, 95%, 96%, 97%, 98% or 100%. The part of the crRNA that interacts with the tracrRNA is preferably at least about 5, 10, 15, 20, 22, 25, 30, 35, 40, 45 or more nucleotides in length. In some embodiments, the part of the crRNA that interacts with the tracrRNA is less than about 60, 55, 50, 45, 40, 35, 30 or 35 nucleotides in length. In an embodiment, the part of the crRNA that interacts with the tracrRNA is about 5-40, 10-35, 15-30, 20-28 nucleotides in length. Preferably, the length of the part that interacts with the tracrRNA is 15, 16, 17, 18, 19, 20, 21, 22, 23, 24, 25, 26, 27, 28, 29, 30, 31, 32, 33, 34 or 35 nucleotides.

The part to the crRNA that interacts with the tracrRNA may be called a linker, which is preferably linked covalently at the 3′ terminus of the crRNA sequence that is complementary to the target DNA sequence (protospacer sequence) and may have at least about 50%, 55%, 60%, 65%, 70%, 75%, 80%, 85%, 90%, 95%, 96%, 97%, 98%, 99% or 100% sequence identity with any one of SEQ ID NO: 17 and 25.

Preferably the tracrRNA, comprises one or more structural motifs that can interact with the CRISPR-system nuclease of the complex as defined herein. Preferably, the tracrRNA is also capable of interacting with the crRNA as defined herein. The tracrRNA and the crRNA may hybridize through base-pairing between the crRNA and the tracrRNA. The tracrRNA preferably is capable of forming a complex with the CRISPR-system nuclease and the crRNA. The crRNA is capable of complexing with the tracrRNA and can hybridize with a target sequence, thereby directing the nuclease to the target sequence.

The tracrRNA may comprise one or more stem-loop structures, such as 1, 2, 3 or more stem loop structures.

The tracrRNA can comprise or consist of non-modified or naturally occurring nucleotides. Alternatively or in addition, the tracrRNA can comprise or consist of modified or non-naturally occurring nucleotides, preferably such chemically modified nucleotides are for protecting the tracrRNA against degradation.

In an embodiment of the invention, the tracrRNA comprises ribonucleotides and non-ribonucleotides. The tracrRNA can comprise one or more ribonucleotides and one or more deoxyribonucleotides.

The tracrRNA may comprise one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-O-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

The tracrRNA may be chemically modified by incorporation of 2′-O-methyl (M), 2′-O-methyl 3′phosphorothioate (MS), 2′-O-methyl 3′thioPACE (phosphonoacetate) (MSP), or a combination thereof, at one or more terminal nucleotides. Such chemically modified tracrRNAs can comprise increased stability and/or increased activity as compared to unmodified tracrRNAs. (Hendel et al, 2015, Nat Biotechnol. 33(9); 985-989). In certain embodiments, a tracrRNA comprises ribonucleotides in a region that interacts with the crRNA.

In an embodiment of the invention, deoxyribonucleotides and/or nucleotide analogues can be incorporated in the engineered tracrRNA structures, such as, without limitation, in the sequence that interacts with the crRNA, in the sequence interacting with the CRISPR-system nuclease or in between these sequences.

Alternatively or in addition, the chemically modified nucleotides can be located 5′ and/or 3′ of the sequence interacting with the crRNA. The chemically modified sequences can further be located 5′ and/or 3′ of the sequence interacting with the CRISPR-system nuclease.

In an embodiment, the length of the tracrRNA can be at least about 25, 30, 35, 40, 45, 50, 55, 60, 65, 70, 72, 75, 80, 85, 90, 95, 100, 110, 120, 130, 140, 150 or more nucleotides in length. In some embodiments, the tracrRNA is less than about 200, 180, 160, 140, 120, 100, 95, 90, 85, 80 or 75 nucleotides in length. Preferably, the length of the tracrRNA is bout 30-120, 40-100, 50-90 or about 60-80 nucleotides in length.

The skilled person understands that the invention is not limited to any specific sequence of the tracrRNA. The tracrRNA may comprise a sequence having at least about 50%, 55%, 60%, 65%, 70%, 75%, 80%, 85%, 90%, 95%, 96%, 97%, 98%, 99% or 100% sequence identity with any one of SEQ ID NO: 18 and 24.

The part of the tracrRNA sequence that interacts with the CRISPR-system nuclease is designed to be sufficient to direct the complexed nuclease to the target sequence. The part of the tracrRNA sequence that interacts with the CRISPR-system nuclease may be at least about 20, 25, 30, 35, 40, 45, 50, 55, 60, 65, 70, 72, 75, 80, 85, 90, 95, 100 or more nucleotides in length. In some embodiments, the sequence interacting with the CRISPR-system nuclease is less than about 120, 100, 80, 72, 70, 60, 55, 50, 45, 40, 30 or 20 nucleotides in length. Preferably, the part of the tracrRNA sequence that interacts with the CRISPR-system nuclease is about 20-90, 30-85, 35-80, 40-75 or 50-72 nucleotides in length. Preferably, the part of the tracrRNA that interacts with the CRISPR-system nuclease is about 40, 42, 44, 46, 48, 50, 52, 54, 56, 58, 60, 62, 64, 66, 68, 70, 72, 74 or 76 nucleotides in length.

The part of the tracrRNA that interacts with the crRNA is designed to be sufficiently complementary to the crRNA to hybridize to the crRNA, and direct the complexed nuclease to the target sequence. Preferably, the complementarity between this part of a tracrRNA sequence and its corresponding part in the crRNA, when optimally aligned using a suitable alignment algorithm, is at least about 50%, 60%, 70%, 80%, 85%, 90%, 91%, 92%, 93%, 94%, 95%, 96%, 97%, 98% or 100%. The part of the tracrRNA that interacts with the crRNA is preferably at least about 5, 10, 15, 20, 22, 25, 30, 35, 40, 45 or more nucleotides in length. In some embodiments, the part of the tracrRNA that interacts with the crRNA is less than about 60, 55, 50, 45, 40, 35, 30 or 35 nucleotides in length. In an embodiment, the part of the tracrRNA that interacts with the crRNA is about 5-40, 10-35, 15-30, 20-28 nucleotides in length. Preferably, the length of the part that interacts with the crRNA is 15, 16, 17, 18, 19, 20, 21, 22, 23, 24, 25, 26, 27, 28, 29, 30, 31, 32, 33, 34 or 35 nucleotides.

### CRISPR-System Nuclease

The CRISPR-system nuclease or the protein of the RNA-guided CRISPR-system nuclease complex for use in the current invention can be any suitable CRISPR-system nuclease that forms a complex with a crRNA and a tracrRNA as defined herein and wherein the complex subsequently can mediate targeted modification of DNA in a plant cell. The CRISPR-system nuclease may be a class 2 CRISPR-nuclease (Makarova K S et al, “An updated evolutionary classification of CRISPR-Cas systems”, Nat. Rev. Microbiol (2015), 13(11):722-726). The CRISPR-system nuclease may be a class 2, type 2 CRISPR nuclease.

The term CRISPR-nuclease, Cas, Cas-protein or Cas-like protein refers to CRISPR related proteins and includes but is not limited to CAS9, CSY4, nickases (e.g. Cas9_D10A, Cas9_H820A or Cas9_H839A), fusion proteins (e.g. Cas9 or Cas-like molecules fused to a further functional domain such as a heterologous nickase/endonuclease domain), and thermostable Cas9 (thermoCas9) nucleases (such as described in e.g. WO2016/198361, WO2018/109101 and WO2018/108339, which are incorporated herein by reference) and other examples, such as for example described in WO2015/006747, WO2018/115390 and U.S. Pat. No. 9,982,279, which are incorporated herein by reference.

Mutants and derivatives of Cas9 as well as other Cas proteins can be used in the methods disclosed herein. Preferably, such other Cas proteins have endonuclease activity and are able to recognize a target nucleic acid sequence when in a cell in the presence of a crRNA and a tracrRNA as defined herein. The CAS-protein or CAS-like protein is preferably a CAS9, or thermostable CAS9, protein. In other embodiments, the Cas protein may be a homolog or ortholog of Cas9, preferably a homolog or ortholog in which at least one of the RuvC, HNH, REC and BH domains is highly conserved.

The CAS or CAS-like protein may be, but is no limited to, selected from the group consisting of: Cas9 from Streptococcus pyogenes (e.g. UniProtKB—Q99ZW2), Cas9 from Francisella tularensis (e.g. UniProtKB—A0Q5Y3), Cas9 from Staphylococcus aureus (e.g. UniProtKB-J7RUA5), Cas9 from Actinomyces naeslundii (UniProtKB—J3F2B0), Cas9 from Streptococcus thermophilus (e.g. UniProtKB—G3ECR1; UniprotKB—Q03J16; Q03LF7), Cas9 from Neisseria meningitidis (e.g. UniProtKB—C9X1G5; UniProtKB—A1IQ68); Listeria innocua (e.g. UniProtKB—Q927P4); Cas9 from Streptococcus mutans (e.g. UniProtKB—Q8DTE3); Cas9 from Pasteurella multocida (e.g. UniProtKB—Q9CLT2); Cas9 from Corynebacterium diphtheriae (e.g. UniProtKB—Q6NKI3); Cas9 from Campylobacter jejuni (e.g. UniProtKB—Q0P897) and Cas9, or “thermoCas9” from Geobacillus thermodenitrificans (e.g. UniProtKB—A0A178TEJ9), any variant or orthologue thereof or any CRISPR associated endonuclease derived therefrom, preferably having at least about 70%, 75%, 80%, 85%, 90%, 95%, 96%, 97%, 98%, 99% or 100% sequence identity with said Cas9 protein.

In an embodiment wherein the CRISPR-system nuclease is a Cas9 nuclease, the PAM sequence preferably comprises at least one of 5′-NGG-3′ and 5′-YG-3′ (see e.g. Hirano et al, Cell 164(5):950-961), wherein N is A, C, G or T and wherein Y is a pyrimidine. Preferably, the CRISPR-system nuclease is a S. pyogenes Cas9 and the PAM sequence comprises 5′-NGG-3′.

A subject nuclease capable of complexing with a tracrRNA can be referred to as a nuclease that is compatible with the tracrRNA. Likewise, a tracrRNA capable of complexing with a CRISPR-system nuclease can be referred to as a nucleic acid compatible with the CRISPR-system nuclease. A subject crRNA capable of complexing with a tracrRNA can be referred to as a nucleic acid compatible with the tracrRNA. Likewise, a tracrRNA capable of complexing with a crRNA can be referred to as a nucleic acid compatible with the crRNA.

Preferably the crRNA and tracrRNA are RNA molecules, but at least one of the crRNA and tracrRNA may also comprise DNA. The CRISPR-system nuclease may form a complex with the tracrRNA and the crRNA, resulting in a RNA-guided CRISPR system nuclease complex.

The CRISPR-system nuclease may comprise endogenous catalytical activity, thereby being capable of introducing a DSB, or may be engineered to comprise at least one catalytically inactive domain, e.g. one active and one inactive domain. The CRISPR-system nuclease may be modified to have one or more inactive catalytic domains, such as an inactive RuvC and/or an inactive HNH domain. For example, the CRISPR-system nuclease for use in the current invention may comprise a RuvC D10A mutation and/or a HNH H840A mutation, or any mutation analogous thereof as compared to said mutation in S. pyogenes Cas9. A CRISPR-system nuclease comprising an active and an inactive domain can also be annotated herein as a nickase. A non-limiting example of a nickase is a Cas9, or thermostable Cas9, comprising a D10A mutation or a H840A mutation. The CRISPR-system nuclease for use in the current invention may comprise a mutation that has the same or a similar effect as a D10A or H840A mutation. In addition or alternatively, the CRISPR-system nuclease for use in the current invention may be any homolog or ortholog of Cas9, or thermostable Cas9, having a mutation at a similar or equivalent position.

The CRISPR-system nuclease protein may contain one or more nuclear localization signal sequences (NLS), mutations, deletions, alterations or truncations. In addition or alternatively, the CRISPR-system nuclease encoding genes may be codon optimized, e.g. for expression in plants.

In an embodiment, the CRISPR-system nuclease does not have any catalytic activity, e.g. due to the presence of a mutation in all catalytic domains, preferably due to an inactivating mutation in the RuvC and HNH domain. Such catalytically inactive CRISPR-system nuclease is also annotated herein as a dead nuclease.

A CRISPR-system nuclease as defined herein includes a catalytically active CRISPR-system nuclease, a partly inactive CRISPR-system nuclease (nickase) and a catalytically inactive CRISPR-system nuclease (dead nuclease), unless indicated otherwise.

An active, partly inactive or dead CRISPR-system nuclease may serve to guide a fused functional domain as detailed herein below to a specific site in the DNA as determined by the crRNA.

Hence in an embodiment, the CRISPR-system nuclease may be fused to a functional domain Optionally, such functional domain is for epigenetic modification, for example a histone modification domain. The domains for epigenetic modification can be selected from the group consisting of a methyltransferase, a demethylase, a deacetylase, a methylase, a deacetylase, a deoxygenase, a glycosylase and an acetylase (Cano-Rodriguez et al, Curr Genet Med Rep (2016) 4:170-179). The methyltransferase may be selected from the group consisting of G9a, Suv39h1, DNMT3, PRDM9 and Dot1L. The demethylase may be LSD1. The deacetylase may be SIRT6 or SIRT3. The methylase may be at least one of KYP, TgSET8 and NUE. The deacetylase may be selected from the group consisting of HDAC8, RPD3, Sir2a and Sin3a. The deoxygenase may be at least one of TET1, TET2 and TET3, preferably TET1cd (Gallego-Bartolomé J et al, Proc Natl Acad Sci USA. (2018); 115(9):E2125-E2134). The glycosylase may be TDG. The acetylase may be p300.

Optionally, the functional domain is a deaminase, or functional fragment thereof, selected from the group consisting of an apolipoprotein B mRNA-editing complex (APOBEC) family deaminase, an activation-induced cytosine deaminase (AID), an ACF1/ASE deaminase, an adenine deaminase, and an ADAT family deaminase. Alternatively or in addition, the deaminase or functional fragment thereof may be ADAR1 or ADAR2, or a variant thereof.

The apolipoprotein B mRNA-editing complex (APOBEC) family of cytosine deaminase enzymes encompasses eleven proteins that serve to initiate mutagenesis in a controlled and beneficial manner. Preferably, the APOBEC deaminase is selected from the group consisting of APOBEC1, APOBEC2, APOBEC3A, APOBEC3B, APOBEC3C, APOBEC3D, APOBEC3F, APOBEC3G, APOBEC3H, APOBEC4 and Activation-induced (cytidine) deaminase. Preferably, the cytosine deaminase of the APOBEC family is activation-induced cytosine (or cytidine) deaminase (AID) or apolipoprotein B editing complex 3 (APOBEC3). These proteins all require a Zn²⁺-coordinating motif (His-X-Glu-X23-26-Pro-Cys-X2_4-Cys) and bound water molecule for catalytic activity. Preferably, in a method of the invention, the deaminase domain fused to the CRISPR-system nuclease is an APOBEC1 family deaminase. Preferably, the deaminase domain is rat deaminase (rAPOBEC1) encoded by a sequence having at least about 80%, 85%, 90%, 95%, 96%, 97%, 98%, 99% or 100% sequence identity with SEQ ID NO: 2 or 3, preferably with SEQ ID NO: 3. In addition or alternatively, the amino acid sequence of the rat deaminase domain has at least about 80%, 85%, 90%, 95%, 96%, 97%, 98%, 99% or 100% sequence identity with SEQ ID NO: 4. Preferably, the deaminase domain has deaminase activity.

Another exemplary suitable type of deaminase domain that may be fused to the CRISPR-system nuclease is an adenine or adenosine deaminase, for example an ADAT family of adenine deaminase. Further, the adenine deaminase may be TadA or a variant thereof, preferably as described in Gaudelli et al., 2017 (Gaudelli et al. 2017 Nature 551: 464-471). Preferably, the deaminase domain is TadA encoded by a sequence having at least about 80%, 85%, 90%, 95%, 96%, 97%, 98%, 99% or 100% sequence identity with SEQ ID NO: 5. In addition or alternatively, the amino acid sequence of TadA has at least about 80%, 85%, 90%, 95%, 96%, 97%, 98%, 99% or 100% sequence identity with SEQ ID NO: 6. Further, the CRISPR-system nuclease may be fused to an adenine deaminase domain, e.g. derived from ADAR1 or ADAR2.

The deaminase domain of the present invention may comprise or consist of a whole deaminase protein or a fragment thereof which has catalytic activity. Preferably, the deaminase domain has deaminase activity.

The functional domain, e.g. the deaminase domain, may be fused to the N- or C-terminus of the CRISPR-system nuclease. Preferably, the functional domain is fused to the N-terminus of the CRISPR-system nuclease. Optionally, the functional domain and the CRISPR-system nuclease used in the method of the invention are fused directly to each other or via a linker. The terms linker and spacer can be used interchangeably herein.

The linker may be any suitable linker known in the art, e.g. ranging from very flexible linkers of the form (GGGGS)n, (GGS)n, and (G)n to more rigid linkers of the form (EAAAK)n (SEQ ID NO: 7), (SPKKKRKVEAS)n (SEQ ID NO: 8), or (SGSETPGTSESATPES)n (SEQ ID NO: 9), or (KSGSETPGTSESATPES)n (SEQ ID NO: 10), or any variant thereof, wherein n preferably is between 1 and 7, i.e. 1, 2, 3, 4, 5, 6, or 7.

The linker preferably has a length between 1 and 32 amino acids, between 2 and 30 amino acids, between 3 and 23 amino acid, and/or between 5 and 18 amino acids.

Optionally, the CRISPR-system nuclease is further fused to an UDG inhibitor (UGI) domain. The UGI domain may be fused to the N- or C-terminus of the CRISPR-system nuclease. Preferably, the UGI domain is fused to the C-terminus of the CRISPR-system nuclease. The fusion may be direct or via a linker as indicated above. Preferably, the CRISSPR-system nuclease is fused to a deaminase domain at the N-terminus of the CRISPR-system nuclease, and the CRISPR-system nuclease is fused to a UGI domain at the C-terminus of the CRISPR-system nuclease.

Uracil DNA glycosylases (UDGs) recognize uracil, inadvertently present in DNA and initiates the uracil excision repair pathway by cleaving the N-glycosidic bond between the uracil and the deoxyribose sugar, releasing uracil and leaving behind a basic site (AP-site). The AP-site is then processed and restored to a canonical base by the subsequent actions of AP-endonuclease, dRPase, DNA polymerase and DNA ligase enzymes. By fusing a UGI domain to the cytosine deaminase containing nuclease fusion protein, the efficiency of base editing increases. Preferably, the UGI domain is or is a variant of UGI from B. subtilis bacteriophage PBS1 or PBS2 (UniProtKB—P14739). Preferably, the nucleotide sequence of the UGI domain may have at least about 80%, 85%, 90%, 95%, 96%, 97%, 98%, 99% or 100% sequence identity with SEQ ID NO: 11 or 12, preferably with SEQ ID NO: 12. Preferably, the amino acid sequence of the UGI domain may have at least about 80%, 85%, 90%, 95%, 96%, 97%, 98%, 99% or 100% sequence identity with SEQ ID NO: 13 or 14, preferably with SEQ ID NO: 14. Preferably, the UGI domain inhibits UDG.

In an embodiment, the UDG inhibitor is not fused to the CRISPR-system nuclease as defined herein, but is contacted to the DNA to be edited as a further functional protein, preferably together with the CRISPR-system nuclease. As a non-limiting example, the cell, preferably the plant cell, may be transfected using the UDG inhibitor or a construct encoding the UDG inhibitor. In the latter case, said construct may further comprise a sequence encoding the CRISPR-system nuclease, or CRISPR-system nuclease fusion protein as defined herein, or alternatively, the UDG inhibitor and CRISPR-system nuclease or CRISPR system nuclease fusion protein may be encoded on separate constructs.

### Nuclease-Assisted Oligonucleotide-Directed Targeted Nucleotide Exchange (Nuclease-Assisted ODTNE)

In an embodiment, the RNA-guided CRISPR-system nuclease complex as defined herein is used for nuclease-assisted ODTNE. Hence, the invention further pertains to a method for targeted modification of DNA in a plant cell, wherein the targeted modification is directed by an oligonucleotide, and wherein the method comprises a step of contacting the DNA with:

i) an RNA-guided CRISPR-system nuclease complex as defined herein; and

ii) an oligonucleotide, preferably an oligonucleotide as defined herein.

Preferably in this embodiment, the nuclease is capable of introducing a single-stranded or double stranded break in the DNA. Preferably, the RNA-guided CRISPR-system nuclease complex as defined herein is capable of introducing a double-stranded break.

In this embodiment, the method of the invention may further comprise a step of introducing into the plant cell a single-stranded oligonucleotide having a sequence that is at least partly complementary to a target sequence. Hence, preferably the oligonucleotide is at least partly complementary to a sequence in the DNA of the plant cell. The single-stranded oligonucleotide is preferably designed such that it comprises a sequence that is capable of hybridizing to the strand of the target DNA opposite to the strand of the target DNA comprising the protospacer sequence. In other words, in case the protospacer sequence is located in the first strand of the duplex DNA, the single-stranded oligonucleotide is to be designed such that it is hybridisable to the second strand of the duplex DNA. Hence in a preferred embodiment, the single-stranded oligonucleotide comprises a sequence that has at least about 70%, 75%, 85%, 90%, 95%, 96%, 97%, 98%, 99% or 100% sequence identity with the protospacer sequence. As indicated further herein, optionally the single-stranded oligonucleotide comprises a sequence that identical to the protospacer sequence with the exception of at least 1, 2 or 3 mismatches.

Such single-stranded oligonucleotide can also be annotated herein as a template oligonucleotide or mutagenic oligonucleotide. Within the context of the current invention “single-stranded” refers to a linear stretch of nucleotides, with a 5′end and a 3′end, without the presence of its fully complementary strand. “Single-stranded” may further refer to a linear stretch of nucleotides, with a 5′ end and a 3′ end, without the presence of a partly complementary strand. A partly complementary strand is defined herein as a strand wherein at least about 15%, 20%, 25%, 30%, 35%, 40%, 45%, 50%, 55%, 60%, 65%, 70%, 75%, 80%, 85%, 90%, 95%, 98%, but less than 100% of the nucleotides are complementary to the oligonucleotide as defined herein. Preferably, the fully or complementary strand has about the same length as the oligonucleotide used in the method of the current invention.

Preferably, the single-stranded oligonucleotide comprises the target sequence except for at least one mismatch with respect to the target sequence. Alternatively or in addition, the single-stranded oligonucleotide may comprise a sequence that is fully complementary to the target sequence, except for at least one mismatch with respect to said complementary sequence. In other words, the single-stranded oligonucleotide comprises the information with respect to the alteration that is to be introduced in the target sequence of the DNA to be modified in the method of the invention. In addition to the target sequence—or its complement—with the one or more mismatches, the single-stranded oligonucleotide, may, in certain embodiment also comprise additional stretches of nucleotides adjacent to the target sequence—or its complement—with the one or more mismatches. However, preferably the oligonucleotide substantially consists of the target sequence—or its complement—with the one or more mismatches relative to the target sequence comprised in a single strand of the DNA duplex. Preferably, the single-stranded oligonucleotide comprises at least one mismatch with respect to at least one of the PAM sequence and protospacer sequence preferably present in the target sequence. Preferably, the single-stranded oligonucleotide comprises at least two or three mismatches with respect to at least one of the PAM and protospacer sequence. Such one or more mismatches will prevent the targeted modified sequence to be retargeted. Preferably, the single-stranded oligonucleotide comprises at least one mismatch with respect to the PAM sequence preferably present in the target sequence, and in addition at least one mismatch outside said PAM sequence. Preferably said at least one mismatch outside said PAM sequence, is a mismatch resulting in a codon alteration, which preferably results in an amino acid change, or early stop, in the encoded protein. The mismatch is preferably at a position where a nucleotide conversion (e.g. a SNP) in the target sequence, or in its complement, is desired. Although the exact position of this mismatch is not crucial, preferably this mismatch is located in the centre part of the ssODN, such as within the central 95%, 90%, 80%, 70%, 60%, 50%, 40%, 30% nucleotides of the ssODN.

In an embodiment of the invention, the crRNA and the mutagenic single-stranded oligonucleotide may comprise a continuous stretch of at least 10, 15, 16, 17, 18, 19, 20, 21, 22, or at least 23 nucleotides having the same sequence, with the exception of one or two mismatches. Alternatively or in addition, the crRNA and the single-stranded oligonucleotide may comprise a continuous stretch of at least 10, 15, 16, 17, 18, 19, 20, 21, 22, or at least 23 nucleotides having the same sequence, with the exception of three or four mismatches.

In an embodiment of the invention, the crRNA and the mutagenic single-stranded oligonucleotide may comprise a continuous stretch of at least 10, 15, 16, 17, 18, 19, 20, 21, 22, or at least 23 nucleotides having a fully complementary sequence, with the exception of one or two mismatches. Alternatively or in addition, the crRNA and the single-stranded oligonucleotide may comprise a continuous stretch of at least 10, 15, 16, 17, 18, 19, 20, 21, 22, or at least 23 nucleotides having a fully complementary sequence, with the exception of three or four mismatches.

The use of ODTNE and the structure and design of the oligonucleotides that are functional in this technology are well described, inter alia in WO98/54330, WO99/25853, WO01/24615, WO01/25460, WO2007/084294, WO2007/073149, WO2007/073166, WO2007/073170 WO2009/002150, WO2012/074385, WO2012/074386, WO2018/115389 and WO2015/139008. The skilled person thus straightforwardly understands how to design a single-stranded oligonucleotide for use in the current invention.

The mutagenic oligonucleotides used in the present invention preferably have a length that is in line with other mutagenic oligonucleotides used in the art, i.e. preferably between about 50 and about 250, more preferably between about 80 and about 220 nucleotides, preferably about 80, 90, 100, 110, 120, 127, 130, 140, 150, 170, 160, 180, 190, 200, 210, or 220 nucleotides.

Preferably, the oligonucleotide comprises at least about 20, 25, 30, 35, 40, 45, 50, 55, 60, 65, 70, 80, 90, or at least about 100 contiguous nucleotides. Preferably the oligonucleotide comprises at least about 20, 25, 30, 35, 40, 45, 50, 55, 60, 65, 70, 80, 90 or at least about 100 contiguous nucleotides that have about 100% sequence identity with respectively at least about 20, 25, 30, 35, 40, 45, 50, 55, 60, 65, 70, 80, 90 or at least about 100 contiguous nucleotides of the DNA, with the exception of one or more, e.g. 2, 3 or 4, mismatches. Preferably, the oligonucleotide comprises at least about 20, 25, 30, 35, 40, 45, 50, 55, 60, 65, 70, 80, 90 or at least about 100 contiguous nucleotides that have about 100% sequence identity with respectively at least about 20, 25, 30, 35, 40, 45, 50, 55, 60, 65, 70, 80, 90 or at least about 100 contiguous nucleotides of the DNA comprising the target sequence, with the exception of one or more, e.g. 2, 3 or 4, mismatches.

The single-stranded oligonucleotide may comprise both RNA and DNA nucleotides, preferably the single-stranded oligonucleotide does not comprise RNA nucleotides. Preferably, the single-stranded oligonucleotide consists of DNA nucleotides. In some embodiments, one or more of the nucleotides of the single-stranded oligonucleotide comprise chemical modifications. Preferably, the single-stranded oligonucleotide is a chemically protected oligonucleotide, preferably comprising at least one, two, three or four chemically protected nucleotides. Such a chemically protected oligonucleotide may be more resistant to nucleases and may in addition provide for higher binding affinity.

The type of modifications to provide a chemically protected oligonucleotide is not in particular limited. Examples of suitable modifications include the introduction of a reverse base (idC) at the 3′ end of the oligonucleotide to create a 3′ blocked end on the repair oligonucleotide; introduction of one or more 2′O-methyl nucleotides or bases which increase hybridization energy (see WO2007/073149) at the 5′ and/or 3′ of the repair oligonucleotide; conjugated (5′ or 3′) intercalating dyes such as acridine, psoralen, and ethidium bromide; introduction of a 5′ terminus cap such as a T/A clamp, a cholesterol moiety, SIMA (HEX), and riboC; backbone modifications such as phosphothioate, methyl phosphonates, MOE (methoxyethyl), di PS and peptide nucleic acid (PNA); or ribose modifications such as 2′ O methyl and locked nucleic acids (LNA). Preferred chemical modifications are either phosphorothioates (PS) that help to protect the single-stranded oligonucleotide from degradation (PS are normally placed at the ends of the single-stranded oligonucleotide) or locked nucleic acids (LNAs) that give both protection against nucleases and also a higher binding affinity (LNAs can be placed either at the ends of the single-stranded oligonucleotide or internally).

Preferably the at least one mismatch is not a chemically protected nucleotide. According to another preference a chemically protected nucleotide is at least one nucleotide from the at least one mismatch. In other words, preferably a mismatch in the single-stranded oligonucleotide is not a chemically protected nucleotide and the nucleotide adjacent to (either side) the mismatch is also not a chemically protected (or modified) nucleotide.

As indicated above, preferably, the ssODN comprises or consists of a contiguous sequence of nucleotides found in plant DNA comprising the target sequence, with the exception of at least one mismatch. The ssODN may be designed such that the arm on the PAM-proximal side of the break (i.e. the arm hybridizing to the sequence adjacent to the break that comprises the PAM sequence or its complement) is about similar in length as the arm on the PAM-distal side of the break (i.e. the arm hybridizing to the sequence adjacent to the break that does not comprise the PAM sequence or its complement). However, preferably, the arm on the PAM-proximal side of the break is at least about twice as long as the arm on the PAM-distal side of the break. More preferably, the arm on the PAM-proximal side of the break is at about three times as long as the arm on the PAM-distal side of the break.

In a preferred embodiment, the single-stranded or mutagenic oligonucleotide is designed as described in Richardson et al, Nature Biotechnology (2016), 43(3), 339-345, which is incorporated herein by reference.

In an embodiment, the at least one mismatch may be located on the PAM-proximal arm. As a non-limiting example, the ssODN may be 127 nucleotides in length, comprising a sequence complementary to the the DNA strand that is not targeted by the sgRNA with a 36 nucleotides arm on the PAM-distal side of the break and a 91 nucleotides arm on the PAM-proximal side of the break. Optionally, the ssODN is modified by having a phosphorothioate linkage between the last 2 nucleotides on each end of the ssODN. Preferably the at least one mismatch for inducing the targeted nucleotide exchange is located within the central about 50% nucleotides of the ssODN, such as within the stretch located at position 30-100 or 32-96 in case the ssODN has a length of 127 nucleotides. Optionally, the ssODN having a length of 127 nucleotides with a 36 nucleotides arm on the PAM-distal side of the break and a 91 nucleotides arm on the PAM-proximal side of the break, has at least one mismatch located at a position between 40-90 from the 5′ end, or at a position 60-80 from the 5′ end, or at position 60, 61, 62, 63, 64, 65, 66, 67, 68, 69, 70, 71, 72, 73, 74, 75, 76, 77, 78, 79 or 80 from the 5′ end.

### Homologous Recombination

In an embodiment, the RNA-guided CRISPR-nuclease complex as defined herein is capable of introducing a single-stranded or double stranded break, e.g. because the nuclease comprises, preferably endogenous, nuclease activity. In this embodiment, the method of the invention may further comprise a step of introducing into the plant cell a donor construct having a sequence that is at least partly complementary to a sequence of the DNA to be modified by the method of the invention. Hence, the invention further pertains to a method for targeted modification of DNA in a plant cell, wherein the targeted modification is directed by a donor construct, and wherein the method comprises a step of contacting the DNA with:

i) an RNA-guided CRISPR-system nuclease complex as defined herein; and

ii) a donor construct, preferably a donor construct as defined herein.

The donor construct is preferably a double-stranded DNA molecule. The double-stranded DNA molecule for use in this embodiment can be a double-stranded oligonucleotide or a longer duplex DNA molecule, such as a DNA fragment. The double-stranded DNA may comprise a 5′ end and a 3′ end. Alternatively, the double-stranded DNA can be circular. As a non-limiting example, the double-stranded DNA molecule can be a plasmid. The size of the donor construct is preferably between about 0.1-10 kb, preferably between about 0.2-8 kb or preferably between about 0.3-7 kb.

In a preferred embodiment, the donor construct comprises at least part of the target sequence. The donor construct preferably further comprises a sequence that is introduced into the plant cell DNA. Preferably, the donor construct comprises a first part of the target sequence, followed by the sequence to be introduced, followed by the second part of the target sequence. In a non-limiting example, the first part of the target sequence may comprise about 15%, 20%, 25%, 30%, 35%, 40%, 45%, 50%, 55%, 60%, 65%, 70%, 75%, 80% or about 85% of the nucleotides that form the target sequence and the second part of the target sequence comprises the remaining part of the target sequence. The target sequence preferably is the same or a similar sequence as the sequence targeted by the crRNA as detailed herein above.

The sequence to be introduced can result in a specific modification, insertion or deletion of the DNA in a plant cell. The modification, insertion or deletion can be a modification, insertion or deletion of one or more nucleotides, such as but not limited to, the modification, insertion or deletion of 2, 3, 4, 5, 10, 20, 30, 40, 50, 100 or more nucleotides.

The donor construct can be used to modify a sequence of the DNA in a plant cell as defined herein, preferably by means of homologous recombination. Hence, the donor construct preferably comprises a sequence to be introduced, wherein said sequence is flanked by sequences that have 100% sequence identity with a sequence in the DNA to be modified. Preferably, the nucleotide sequence of these flanking sequences are identical to respectively the 5′ and 3′ sequence of the DNA located immediately adjacent to the cleavage site generated by the RNA-guided CRISPR-system nuclease complex.

In the presence of the homologous sequence comprised in the donor construct, the DSB can be repaired by homologous recombination (HR). This is the basis for the process of gene targeting whereby, rather than a sister chromatid being used for repair, information is copied from the donor construct that is introduced into the cell.

The donor construct comprises alterations or an exogenous sequence compared with the original chromosomal locus, and thus the process of HR incorporates these alterations or exogenous sequence in the genome. Preferably, the exogenous sequence to be introduced by HR is located within the donor construct in between the sequences at its 3′-end and at its 5′-end that has 100% sequence identity or 100% sequence complementarity with a sequence in the DNA.

The donor construct can be linearized or circular. Preferably, the donor construct is a linearized molecule. The first and/or second strand of the double-stranded DNA molecule can comprise a chemical modification, e.g. to make the molecule at least partly resistant to nucleases. Preferably, the chemical modification is a modification as defined herein above. Preferably, the donor construct comprises RNA, DNA or is an RNA-DNA hybrid. Preferably, the donor construct comprises DNA.

### Introducing the RNA-Guided CRISPR-System Nuclease Complex

The method of the invention preferably comprises a step of contacting the DNA of the plant cell with an RNA-guided CRISPR-system nuclease complex as defined herein. This may be accomplished by introducing into the plant cell a CRISPR-system nuclease, a tracrRNA and a crRNA for guiding the nuclease to the DNA. The method of the invention may therefore also be defined as a method for targeted modification of DNA in a plant cell, wherein the method comprises a step of introducing into the plant cell a RNA-guided CRISPR-system nuclease complex as defined herein.

The CRISPR-system nuclease may be delivered into the plant cell in the form of at least one of a CRISPR-system nuclease protein, an mRNA or RNA encoding the CRISPR-system nuclease protein, and/or a vector comprising a gene encoding the CRISPR-system nuclease.

The tracrRNA may be delivered into the plant cell in the form of a tracrRNA molecule and/or a vector encoding the tracrRNA.

The crRNA may be delivered into the plant cell in the form of at least one of a crRNA molecule, a precursor crRNA molecule and a vector encoding the crRNA molecule.

In an embodiment, the CRISPR-system nuclease complex is introduced into the cell as pre-assembled ribonucleo-protein complex, i.e. comprising a CRISPR-system nuclease, a tracrRNA and a crRNA. Alternatively or in addition, the plant cell can be transfected with one or more vectors encoding the CRISPR-system nuclease, crRNA and tracrRNA.

In an embodiment, at least one of the components of the RNA-guided CRISPR-system nuclease complex can be introduced directly into the cell, while another component can be expressed from a vector. As a non-limiting example, the CRISPR-system nuclease may be expressed from a vector, while the tracrRNA and/or crRNA can be introduced directly into the plant cell.

In an embodiment, one or more of the components of the RNA-guided CRISPR-system nuclease complex as defined herein is stably expressed in the plant cell. In this embodiment, at least one of the CRISPR-system nuclease, the tracrRNA and the crRNA is stably expressed in the plant cell. As a non-limiting example, a CRISPR-system nuclease as defined herein can be stably expressed in the plant cell, while the tracrRNA and/or crRNA can be transiently introduced, e.g. as crRNA/tracrRNA molecules and/or as a vector(s) expressing the crRNA and/or tracrRNA.

In an embodiment, introduction of a vector results in the transient expression of the encoded nuclease or of the encoded crRNA or tracrRNA molecule.

The introduction in the plant cell or transfection may be performed by any conventional method known in the art. Optionally, sequences encoding the CRISPR system nuclease and/or crRNA and/or tracrRNA are stably introduced in the genome of the plant cell. In case the CRISPR-system nuclease complex is delivered in the cell as a ribonucleo-protein complex, the method of the invention further comprises the step of forming said complex prior to the step of introducing said complex in the plant cell.

Optionally, the method further comprises a step of introducing a single-stranded oligonucleotide for nuclease-assisted ODTNE as defined herein, or a step of introducing a donor construct for HR as defined herein.

Optionally, the RNA-guided CRISPR-system nuclease complex, or one or more vectors encoding one or more components of the RNA-guided CRISPR-system nuclease complex, and optionally the single-stranded oligonucleotide for nuclease-assisted ODTNE or the donor construct for HR, are introduced in the plant cell in a single step, i.e. at substantially the same time. In other words, preferably the CRISPR-system nuclease protein or construct encoding the protein, the crRNA or construct encoding the crRNA, the tracrRNA or construct encoding the tracrRNA and optionally the single-stranded oligonucleotide for nuclease-assisted ODTNE or donor construct for HR, are introduced in the plant cell in a single transfection step. Optionally, these components are transfected in two or more transfections steps.

Optionally, one type of CRISPR-system nuclease, one type of tracrRNA and two or more types of crRNAs are introduced into the plant cell, wherein each type of crRNA comprises a different guide sequence, i.e. targets a different sequence within the plant genome.

In a preferred embodiment, the cell is transformed with at least one CRISPR-system nuclease, i.e., the CRISPR-system nuclease protein is delivered directly into the cell. In a further embodiment, the cell is transformed with at least one crRNA and/or at least one tracrRNA.

Preferably, the method of the invention comprises a step of complexing the CRISPR-system nuclease protein with the tracrRNA and at least one crRNA, before introducing the formed RNA-guided CRISPR-system nuclease complex in the cell. Put differently in this embodiment, the RNA-guided CRISPR-system nuclease complex is introduced in the plant cell as a pre-formed complex. Optionally, said introduction step also comprises the introduction of a single-stranded oligonucleotide for nuclease-assisted ODTNE as defined herein, or a double-stranded donor construct for HR as defined herein.

In another preferred embodiment, the cell is transfected with a vector encoding at least one CRISPR-system nuclease. The cell may further be transfected with an additional vector encoding a tracrRNA and a vector encoding at least one crRNA, wherein optionally, said vector encodes for two or more crRNAs, wherein each crRNA comprises a different guide sequence, i.e. targets a different sequence within the plant genome.

Preferably, within this embodiment, the cell is transfected with a single construct encoding at least one tracrRNA and one or more crRNAs as defined herein. Preferably, within this embodiment, the cell is transfected with a single construct encoding at least one CRISPR-system nuclease, one tracrRNA and one or more crRNAs as defined herein.

A vector encoding at least one of the CRISPR-system nuclease, crRNA and tracrRNA may further comprise transcription regulatory sequences and the gene or genes may be driven by either a constitutive, inducible, tissue-specific or species-specific promoter when applicable, preferably suitable for expression in plant cells. Preferably, expression of a CRISPR-system nuclease may be controlled by a constitutive, inducible, tissue-specific or species-specific promoter that is suitable for expression in plant cells.

Preferably, the nucleotide sequence encoding the CRISPR-system nuclease protein, the nucleotide sequence encoding the crRNA and the nucleotide sequence encoding the tracrRNA are under control of a promoter. The promoter controlling the expression of the CRISPR-system nuclease, the promoter controlling the expression of the tracrRNA and the promoter controlling the expression of the crRNA can be different promoter types. Preferably the promoter controlling the expression of the crRNA and the promoter controlling the expression of the tracrRNA is different from the promoter controlling the expression of the CRISPR-system nuclease.

As a non-limiting example, the CRISPR-system nuclease may, preferably, be under control of a constitutive promoter, preferably suitable for expression in plant, such as the 35 S promoter (e.g. the 35 S promoter from cauliflower mosaic virus (CaMV; Odell et al. Nature 313:810-812; 1985). Other suitable constitutive promoters include, but are not limited to, the cassava vein mosaic virus (CsVMV) promoter, and the sugarcane bacilliform badnavirus (ScBV) promoter (see e.g. Samac et al. Transgenic Res. 2004 August; 13(4):349-61.) Other constitutive promoters include, for example, the core promoter of the Rsyn7 promoter and other constitutive promoters disclosed in WO 99/43 838 and U.S. Pat. No. 6,072,050; ubiquitin (Christensen et al., Plant Mol. Biol. 12:619-632, 1989 and Christensen et al., Plant Mol. Biol. 18:675-689, 1992); pEMU (Last et al., Theor. Appl. Genet. 81:581-588, 1991); AA6 promoter (WO2007/069894); and the like.

The vector may also include transcription termination regions. Where transcription termination regions are used, any termination region may be used in the preparation of the vectors. Exemplary transcript termination and polyadenylation signals are either NosT, RBCT, HSP18.2T or other gene specific or species-specific terminators. The CRISPR-system nuclease gene cassettes or the RNA encoding a CRISPR-system nuclease may contain introns, either native or artificially introduced introns.

In a preferred embodiment, the vector is for transient expression. In other words, the expression in the plant material is temporary as a consequence of the non-permanent presence of the vector. Expression may, for instance, be transient when the construct is not integrated into the host genome. For example, CRISPR-system nuclease protein, crRNA and tracrRNA may be transiently provided to a plant cell, followed by a decline in the amount of at least one of the CRISPR-system nuclease, crRNA and tracrRNA. Subsequently, the plant cell, progeny of the plant cell, and/or plants which comprise the plant cell or have been derived from the plant protoplast wherein the duplex DNA has been altered, comprise a reduced amount of one or more of these components used in the method of the invention, or no longer contain one or more of the components.

Preferably, said plant cell, progeny of the plant cell, and/or plants which comprise the plant cell or have been derived from the plant protoplast wherein the duplex DNA has been altered does not comprise at least one of the CRISPR-guided nuclease, crRNA and tracrRNA. Preferably, said plant cell, progeny of the plant cell, and plants which comprise the plant cell or have been derived from the plant protoplast wherein the duplex DNA has been altered, still comprise the DNA modification.

The vector encoding the CRISPR-system nuclease may be optimized for increased expression in the transformed plant, i.e. codon-optimized for expression in the plant cell. For instance, the nucleotide sequence encoding the CRISPR-system nuclease may be codon-optimized for expression in tomato, wherein said tomato preferably is Solanum lycopersicum. That is, the vector encoding the CRISPR-system nuclease can be synthesized using plant-preferred codons for improved expression. See, for example, Campbell and Gowri, (Plant Physiol. 92: 1-11, 1990) for a discussion of host-preferred codon usage. Methods are available in the art for synthesizing plant-preferred genes (see, for example, Murray et al., Nucleic Acids Res. (1989) 17:477-498, or Lanza et al. (2014) BMC Systems Biology 8:33-43), which are incorporated herein by reference.

There are many suitable approaches known in the art for delivering the nucleic acids (encoding the CRISPR-system nuclease and/or (encoding) the tracrRNA and/or crRNA) or the proteins or ribonucleo-protein complexes into the cell. The delivery system may for example constitute a viral-based delivery system or a non-viral delivery system.

Non-limiting examples of non-viral delivery systems include chemical-based transfection (e.g. using calcium phosphate, dendrimers, cyclodextrin, polymers, liposomes, or nanoparticles), non-chemical-based methods (e.g. electroporation, cell squeezing, sonoporation, optical transfection, protoplast fusion, impalefection, heat shock and hydrodynamic delivery), particle-based methods (e.g. a gene gun or magnet-assisted transfection) and bacterial-based delivery systems (e.g. agrobacterium-mediated delivery). A non-limiting example of a viral delivery system includes the Tobacco Rattle Virus.

In a preferred embodiment, the nucleic acids and/or proteins are introduced into the cell using an aqueous medium, wherein the aqueous medium comprises PEG. Any suitable method can be used, preferably the medium has a pH value of between 5-8, preferably between 6-7.5. Next to the presence in the aqueous medium of the CRISPR-system nuclease and optionally the tracrRNA and/or crRNA, the medium comprises polyethylene glycol. Polyethylene glycol (PEG) is a polyether compound with many applications from industrial manufacturing to medicine. PEG is also known as polyethylene oxide (PEO) or polyoxyethylene (POE). The structure of PEG is commonly expressed as H—(O—CH2-CH2)n-OH. Preferably, the PEG used is an oligomer and/or polymers, or mixtures thereof with a molecular mass below 20,000 g/mol.

The aqueous medium comprising the population of e.g. plant cells preferably comprises 100-400 mg/ml PEG. So the final concentration of PEG is preferably between 100-400 mg/ml, for example, between 150 and 300 mg/ml, for example between 180 and 250 mg/ml. A preferred PEG is PEG 4000 Sigma-Aldrich no. 81240. (i.e. having an average Mn 4000 (Mn, the number average molecular weight is the total weight of all the polymer molecules in a sample, divided by the total number of polymer molecules in a sample.). Preferably the PEG used as a Mn of about 1000-10 000, for example between 2000-6000).

In a further preferred embodiment, the aqueous medium comprising PEG does not comprise more than about 0.001%, 0.01%, 0.05%, 0.1%, 1%, 2%, 5%, 10% or 20% (v/v) glycerol. Preferably, the medium comprises less than about 0.001%, 0.01%, 0.05%, 0.1%, 1%, 2%, 5%, 10% or 20% (v/v) glycerol. In particular for the introduction of a CRISPR-system nuclease protein, the aqueous medium comprises less than about 0.1% (for example, less than 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% (v/v) glycerol. Optionally, the aqueous medium comprising the population of plant cells is completely free of glycerol.

Preferably, the cell cycle of e.g. plant cells is synchronized when exposing the duplex DNA to the RNA-guided CRISPR-system nuclease complex. The synchronization preferably takes places when the RNA-guided CRISPR-system nuclease complex or nucleic acid(s) encoding the same is introduced into the cell as detailed herein. Synchronization is preferably performed by contacting the (plant) cell with a synchronizing agent.

Such method of synchronizing the cell cycle of the (plant) cell has been described in detail in European patent EP2516652, incorporated herein by reference. More particular, synchronizing the (plant) cells, for example, the plant protoplasts may be advantageous in certain embodiments of the invention to further enhance efficacy of the introduction of the alteration in the duplex DNA. Thus, in certain embodiments, the method comprises a step of synchronizing the cell cycle of the cell, preferably a plant cell.

The synchronization preferably takes places when the RNA-guided CRISPR-system nuclease complex or nucleic acid(s) encoding the same is introduced into the cell as detailed herein, such that most of the (plant) cells will be in the same phase of the cell cycle when the duplex DNA is exposed to the site-specific nucleases as defined herein. This may be advantageous and increase the rate of introduction of the alteration in the duplex DNA.

Synchronizing the (plant) cell may be accomplished by any suitable means. For example, synchronization of the cell cycle may be achieved by nutrient deprivation such as phosphate starvation, nitrate starvation, ion starvation, serum starvation, sucrose starvation, auxin starvation.

Synchronization can also be achieved by adding a synchronizing agent to the (plant) cell. Preferably, the synchronizing agent is selected from the group consisting of aphidocolin, hydroxyurea, thymidine, colchicine, cobtorin, dinitroaniline, benefin, butralin, dinitramine, ethalfluralin, oryzalin, pendimethalin, trifluralin, amiprophos-methyl, butamiphos dithiopyr, thiazopyr propyzamide, tebutam DCPA (chlorthal-dimethyl), mimosine, anisomycin, alpha amanitin, lovastatin, jasmonic acid, abscisic acid, menadione, cryptogeine, hydrogenperoxide, sodiumpermanganate, indomethacin, epoxomycin, lactacystein, icrf 193, olomoucine, roscovitine, bohemine, staurosporine, K252a, okadaic acid, endothal, caffeine, MG 132, cycline dependent kinases and cycline dependent kinase inhibitors, as well as their target mechanism. The amounts and concentrations and their associated cell cycle phase are described for instance in “Flow Cytometry with plant cells”, J. Dolezel c.s. Eds. Wiley-VCH Verlag 2007 pp 327 ff. Preferably, the synchronizing agent is aphidicolin and/or hydroxyurea.

Preferably, in the method of the invention, synchronizing the cell cycle synchronizes the (plant) cell in the S-phase, the M-phase, the G1 and/or G2 phase of the cell cycle.

In a preferred embodiment, the CRISPR-system nuclease comprises two catalytically active endonuclease domains. Within this embodiment, the RNA-guided CRISPR-system nuclease complex will introduce a double-strand break in the target sequence. Subsequent activation of the repair mechanism results in alteration of the target sequence of the plant genome. The targeted alteration may comprise the insertion, deletion or modification of at least one base pair. For example, the targeted alteration may comprise the deletion of at least one base pair and the insertion of at least one base pair. For example, 1, 2, 3, 4, 5, 6, 7, 8, 9, 10 or more base pairs may be altered with the method of the invention. More than one modification may be introduced in a single experiment, and/or the experiment may be repeated to introduce subsequent alterations in the genome of the plant cell, optionally at other or at the same gene as targeted in the first event.

### Plant Cell

The method of the invention may further comprise the step of providing a plant cell, preceding the step of introducing into said plant cell a RNA-guided CRISPR-system nuclease complex as defined herein. The skilled person understands that the method of the invention is not limited to a certain plant cell type. In particular, the method of the invention as disclosed herein can be applied to dividing as well as non-dividing cells. The cell may be transgenic or non-transgenic. The plant cell can for example be obtainable from plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grains and the like.

In a preferred embodiment, the plant cell is a plant protoplast. The skilled person is aware of methods and protocols for preparing and propagating plant protoplasts, see for example Plant Tissue Culture (ISBN: 978-0-12-415920-4, Roberta H. Smith). The plant protoplast for use in the method of the current invention can be provided using common procedures used for the generation of plant cell protoplasts (e.g. the cell wall may be degraded using cellulose, pectinase and/or xylanase).

Plant cell protoplasts systems have for example been described for tomato, tobacco and many more (Brassica napus, Daucus carota, Lactucca sativa, Zea mays, Nicotiana benthamiana, Petunia hybrida, Solanum tuberosum, Oryza sativa). The present invention is generally applicable to any protoplast system, including those, but not limited to, the systems described in any one of the following references: Barsby et al. 1986, Plant Cell Reports 5(2): 101-103; Fischer et al. 1992, Plant Cell Rep. 11(12): 632-636; Hu et al. 1999, Plant Cell, Tissue and Organ Culture 59: 189-196; Niedz et al. 1985, Plant Science 39: 199-204; Prioli and Söndahl, 1989, Nature Biotechnology 7: 589-594; S. Roest and Gilissen 1989, Acta Bot. Neerl. 38(1): 1-23; Shepard and Totten, 1975, Plant Physiol. 55: 689-694; Shepard and Totten, 1977, Plant Physiol. 60: 313-316, which are incorporated herein by reference.

The plant cell is preferably obtainable from a crop plant such as a monocot or dicot or of a crop or grain plant such as cassava, corn, sorghum, soybean, wheat, oat or rice. A crop plant is a plant species which is cultivated and bred by humans. A crop plant may be cultivated for food purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork and the like.

The plant cell may also be of an alga, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; plants of the genus Solanum, preferably Solanum lycopersicum).

In another preferred embodiment, the cell is obtainable from a plant selected from the group consisting of asparagus, barley, blackberry, blueberry, broccoli, cabbage, canola, carrot, cassava, cauliflower, chicory, cocoa, coffee, cotton, cucumber, eggplant, grape, hot pepper, lettuce, maize, melon, oilseed rape, pepper, potato, pumpkin, raspberry, rice, rye, sorghum, spinach, squash, strawberry, sugar cane, sugar beet, sunflower, sweet pepper, tobacco, tomato, water melon, wheat, and zucchini.

Preferably, the obtained plant cell comprising the targeted alteration is regenerated into a plant or descendent therefore. Therefore a preferred embodiment of the invention, the method further comprises a step of regenerating a plant or descendent thereof comprising the targeted alteration.

### Plant Cell and Plant or Plant Products

The method may further comprise the step of regenerating a plant or descendant thereof comprising the targeted modification. Preferably, such regeneration is performed using conditions suitable for regeneration. The skilled person is well aware of methods and protocols of regenerating a plant from a plant protoplast. Progeny, descendant's, variants, and mutants of the regenerated plants are also included within the scope of the invention, provided that these parts comprise the targeted alteration introduced with the method taught herein.

In addition to the plant cell or plant comprising the targeted modification, the invention also pertains to a plant cell transiently or stably expressing a CRISPR-system nuclease complex as defined herein. Preferably, the plant cell is a transgenic plant modified to comprise a sequence encoding a CRISPR-system nuclease in its genome as defined herein. In addition, the transgenic plant cell may further comprise transiently or stably expressing a tracrRNA and crRNA as defined herein.

Included within the scope of the invention is such transgenic plant cell and plant regenerated thereof, any progeny, descendant's, variants, and mutants comprising the sequence encoding the CRISPR-system endonuclease, preferably under the control of an inducible promoter and/or a meristem promoter which may be constitutive active meristem promoter, and further comprising a tracrRNA and a crRNA. The invention therefore also pertains to a method for producing such transgenic plant cell and/or plant derived therefrom comprising the step of integrating into its genome a CRISPR-system nuclease encoding sequence as defined herein and a step of introducing a crRNA and tracrRNA as defined herein.

The cell or organism obtainable by a method of the invention may subsequently be propagated to e.g. obtain a culture of cells, (part of) an organism or any descendants thereof.

Preferably, the cell is a plant cell obtainable from a crop plant such as a monocot or dicot or of a crop or grain plant such as cassava, corn, sorghum, soybean, wheat, oat or rice. The plant cell may also be of an alga, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; plants of the genus Solanum, preferably Solanum lycopersicum).

In another preferred embodiment, the cell is obtainable from a plant selected from the group consisting of Arabidopsis, asparagus, barley, blackberry, blueberry, broccoli, cabbage, canola, carrot, cassava, cauliflower, chicory, cocoa, coffee, cotton, cucumber, eggplant, grape, hot pepper, lettuce, maize, melon, oilseed rape, pepper, potato, pumpkin, raspberry, rice, rye, sorghum, spinach, squash, strawberry, sugar cane, sugar beet, sunflower, sweet pepper, tobacco, tomato, water melon, wheat, and zucchini.

The invention also pertains to the progeny of a plant cell or plant obtainable by a method of the invention. Further, the invention pertains to a plant product obtainable from the plant cell or plant as defined herein, e.g. selected from the group consisting of fruits, leaves, plant organs, plant fats, plant oils, plant starch, and plant protein fractions, either crushed, milled or still intact, mixed with other materials, dried, frozen, and so on. These products may be non-propagating. Preferably, said plant product comprises at least or at least part of one of:

i) the modified DNA, preferably the modified GOI as defined herein, or encoded products thereof,

ii) the tracrRNA as defined herein;

iii) the crRNA as defined herein;

iv) the CRISPR-system nuclease as defined herein; and

v) the CRISPR-system nuclease encoding sequence.

Preferably, these products comprise at least fractions of the modified DNA and at least one of the crRNA and tracrRNA, which allows to assess that the plant product is derived from a plant obtained by a method as defined herein.

### Kit of Parts

The invention further concerns a kit of parts, preferably a kit of parts for use in the method as described herein. Preferably, the kit of parts comprises at least one of:
A container comprising a CRISPR-system nuclease as defined herein;
A container comprising a crRNA as defined herein and a container comprising tracrRNA as defined herein. Optionally, the crRNA and tracrRNA can be combined in a single container. The container may further optionally comprise two or more crRNAs, each comprising different guide sequences for targeting different target sequences;
A container comprising single-stranded oligonucleotide for nuclease-assisted ODTNE as defined herein;
A container comprising a double-stranded oligonucleotide for HR as defined herein
A container comprising a vector encoding the CRISPR-system nuclease as defined herein. Optionally, said vector further encodes one or more crRNAs and/or tracrRNAs for guiding said nuclease as defined herein; and
A container comprising one or more vectors encoding one or more crRNAs as defined herein. The one or more vectors may further comprise a sequence encoding a tracrRNA. Alternatively or in addition, the sequence encoding the tracrRNA can be present on a different vector. Said vector encoding the tracrRNA can be present in the same container or in a separate container;
   or any combination thereof. Preferably, the kit of parts comprises a set of containers, wherein the set of containers comprise at least:
a CRISPR-system nuclease as defined herein, or sequence encoding the CRISPR-system nuclease;
a tracrRNA as defined herein, or sequence encoding the tracrRNA; and
a crRNA as defined herein, or sequence encoding the crRNA.
   The set of containers optionally further comprises
a single-stranded oligonucleotide for nuclease-assisted ODTNE as defined herein; and
a double-stranded oligonucleotide for HR as defined herein
   The kit of parts may further comprise a container comprising one or more substances for transfection as defined herein. Also included within the kit may be a manual for performing the method of the invention of modifying a DNA within the plant cell as specified herein.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

### Further Aspects

The invention further pertains to a composition comprising one or more tracrRNAs and one or more, preferably two or more crRNAs as defined herein. Preferably, the crRNA, or crRNAs, is/are characterized in that the guide sequence is for targeting a sequence in the plant genome, preferably a sequence within the GOI as defined herein. The composition may further comprise a CRISPR-system nuclease and/or a nucleic acid encoding a CRISPR-system nuclease as defined herein.
The invention further pertains to nucleic acids and/or vectors encoding one or more tracrRNAs and one or more crRNAs as defined herein, which crRNAs are characterized in that the guide sequence is for targeting a sequence in the plant genome, preferably a sequence within the GOI as defined herein. Optionally, the nucleic acid and/or vector further comprises a sequence encoding a CRISPR-system nuclease as defined herein. Preferably, the sequence encoding the CRISPR-system nuclease is codon optimized for expression in plant cells. Such nucleic acids or vectors may be single-stranded, double stranded, or partially double-stranded; nucleic acid molecules that comprise one or more free ends, no free ends (e.g., circular); nucleic acid molecules that comprise DNA, RNA, or both; and other varieties of polynucleotides known in the art.
The invention also pertains to the use of a RNA-guided CRISPR-system nuclease complex as defined herein, or one or more nucleic acids encoding the same, in a method of the invention, i.e. for use in modifying a plant cell DNA.
The invention further pertains to the use of a RNA-guided CRISPR-system nuclease complex as defined herein, or one or more nucleic acids encoding the same, for stable expression a plant cell.
The invention further pertains to a method for targeted alteration of a coding sequence (CDS) in duplex DNA, preferably as described in PCT/EP2018/074150 which is incorporated herein by reference, wherein the method comprises a step of exposing the duplex DNA to at least two site-specific RNA-guided CRISPR-system nuclease complexes, wherein a first site-specific nuclease complex cleaves the DNA generating a first indel at a first location within the ORF and wherein a second site-specific nuclease complex cleaves the DNA generating a second indel at a second location within the same CDS, wherein the CDS before the first indel and after the second indel remain in the same reading frame, and wherein the altered CDS does not comprise a stop codon.
Having now generally described the invention, the same will be more readily understood through reference to the following example which is provided by way of illustration and is not intended to be limiting of the present invention.

### Examples

The performances of single guide RNA (sgRNA) vs. dual guide RNA (dgRNA, i.e. separate crRNA and tracrRNA molecule) in editing a fluorescent reporter gene in Arabidopsis protoplasts was evaluated in plants.

### Arabidopsis Protoplasts

A reporter construct consisting of a DsRED open reading frame fused to a disrupted YFP open reading frame containing an A to C transversion at position 123 resulting in a premature stop codon was cloned under the control of the CaMV 35S promoter. A 6×HIS tag and a nuclear localization signal (NLS) were attached to the 5′-end of the DsRED ORF and the NOS terminator was use to terminate transcription. The open reading frame of the NLS-His-tagged-DsRED::YFP stop is represented by SEQ ID NO: 15, see below. The reporter cassette was cloned into pK2GW7 using Gateway technology and transformed in Arabidopsis using the floral dip method (Clough S J and Bent A F (1998) Floral dip: a simplified method for agrobacterium-mediated transformation of Arabidopsis thaliana. Plant J 16(6):735-473). Primary transformants were selected on the basis of DsRED fluorescence in protoplasts isolated from young leaves.

### Guide RNAs

sgRNA and dgRNA were designed to comprise the 20 nucleotide-long gene specific sequence 5′-TCTGTTTCTGGTGAGGGTGA-3′ (SEQ ID NO: 16) for targeting a DsRED::YFP stop fluorescent reporter construct stably integrated in a transgenic Arabidopsis line.

For comparison of sgRNA vs. dgRNA, a sgRNA comprising the 20 nt gene specific sequence was purchased from Synthego (Menlo Park, Calif., USA), which further comprises a 80 nt scaffold at the 3′ end of the gene specific sequence. For the dgRNA, a crRNA, comprising a 22nt linker appended to the 3′-end of the 20nt long gene specific sequence, and a 72 nt tracrRNA were purchased from Synthego (Menlo Park, Calif., USA). An sgRNA comprising the 20 nt gene specific sequence and an 80 nt scaffold at the 3′ end of the gene specific sequence was purchased from Synthego (Menlo Park, Calif., USA). An example of a suitable sequence for use as a crRNA linker is: 5′-GUUUUAGAGCUGUGUUGUUUCG-3′ (SEQ ID NO: 17), an example of a suitable sequence for use as a tracrRNA is: 5′-CGAAACAGCAUAGCAAGUUAAAAUAAGGCUAGUCCGUUAUCAACUUGAAAAAGUGGCAC CGAGUCGGUGCUUU-3′ (SEQ ID NO: 18), and an example of a suitable sequence for use as a sgRNA scaffold is: 5′-GUUUUAGAGCUAGAAAUAGCAAGUUAAAAUAAGGCUAGUCCGUUAUCAACUUGAAAAA GUGGCACCGAGUCGGUGCUUUU-3′ (SEQ ID NO: 19) (e.g. see Jinek et al. Science 2012, 337 (6096): 816-821; Cong et al. Science 2013, 339 (6121):819-823; and Zhou et al. Nucleic Acids Res. 2014, 42(17): 10903-10914).

For comparison of synthetic sgRNA vs in vitro transcribed sgRNA, in vitro transcribed sgRNA was made using the EnGen® sgRNA synthesis kit (#E33225 New England BioLabs, Inc, Ipswich, Mass., USA), having the following final sequence:

(SEQ ID NO: 20)
5′-GUCUGUUUCUGGUGAGGGUGACGUUUUAGAGCUAGAAAUAGCAAGUU
AAAAUAAGGCUAGUCCGUUAUCAACUUGAAAAAGUGGCACCGAGUCGGUG
CUUUU-3′.

### Ribonucleoprotein Assembly

The crRNAs and tracrRNAs were annealed according to the manufacturer's instructions (Synthego Corporation, Menlo Park, Calif., USA). In brief, dry crRNA and tracrRNA were dissolved in 1×TE buffer to a final concentration of 200 μM and crRNA and tracrRNAs were mixed in a 2:1 ratio in 1× annealing buffer to a final concentration of 30 μM of annealed dgRNA.

Ribonucleoprotein (RNP) assembly was performed as follows: sgRNA or annealed cr/tracrRNA were diluted in 1×Cas9 reaction buffer (#M0386M, New England BioLabs, Inc, Ipswich, Mass., USA) to a final concentration of 200 ng·μL-1. Cas9 protein (#M0386M, New England BioLabs, Inc, Ipswich, Mass., USA) was added to a final concentration of 1 μg·μL-1. The mixture was briefly mixed by vortexing and pulse-spun in a table centrifuge. Ribonucleoprotein formation was allowed to proceed for 10 minutes at room temperature. Assembled ribonucleoproteins were then stored at 4° C. until use. For each transfection of 250.000 protoplasts, 20 μL of pre-assembled ribonucleoproteins with a 1:1 molar ratio of Cas9:sgRNA were used.

### Single-Stranded Oligonucleotide

A single-stranded oligonucleotide (ssODN) was designed according to Richardson et al, Nature Biotechnology (2016), 43(3), 339-345. In brief the ssODN consists of a sequence complementary to the DNA strand that is not targeted by the sgRNA with a 36 nucleotides arm on the PAM-distal side of the break and a 91 nucleotides arm on the PAM-proximal side of the break, and modified by having a phosphorothioate linkage between the last 2 nucleotides on each end of the ssODN. The ssODN possessed a G at position 73 from the 5′-end for introduction of a C in the upper DNA strand (comprising the open reading frame) converting the TAA (stop) into a TAC (TYR) and consequently restoring the YFP open reading frame. The sequence of the ssODN is as follows:

(SEQ ID NO: 21)
A*GTAGTAACAAGAGTTGGCCATGGAACTGGAAGCTTTCCAGTAGTGCAG
ATGAACTTAAGTGTAAGTTTACCgTAAGTAGCATCACCTTCtCCtTCtCC
tGAAACAGAGAACTTATGTCCGTTCAC*A,

wherein the lowercase characters representing SNPs with respect to the target sequence, all except for the underlined one, are synonymous mutations that do not change the amino acid sequence of the YFP but change the sequence of the protospacer to avoid re-cutting by the Cas9 after gene targeting has occurred. The underlined corresponds to the causative mutation that restores YFP functionality upon gene targeting. Asterisks correspond to phosphorothioate linkages.

The ssODN comprising the sequence and having the indicated modification was ordered from Eurogentec (Liége, Belgium). SDS-PAGE purified ssODNs were dissolved in nuclease-free water to a final concentration of 1 μg·μL-1, heated for 10 minutes at 90° C., pulse-spin for a few seconds and stored at −20° C.

### Protoplasts Transfection and Cultivation

Arabidopsis protoplasts were transfected with RNPs prepared as indicated above with or without ssODN using PEG-mediated transformation as described in EP2562261, which is incorporated herein by reference. In brief, 20 μL pre-assembled RNPs prepared as indicated above was added to a 250 μL aliquot of 250.000 Arabidopsis protoplasts, with or without 10 μL of ssODN (Stock 1 μg·μL-1 in nuclease-free water). The aliquot is subsequently gently admixed with 250 μL PEG. After 20 minutes incubation at room temperature, 5 mL cold 0.275 M Ca(NO₃)₂ are added dropwise. The protoplast suspension is centrifuged for 10 min at 85×g at 4° C.

The supernatant was discarded and the pellet of 250.000 cells re-suspended in 1 mL of B5 medium supplemented with 0.4M glucose, 1 mg·L-1 NAA, 0.2 mg·L-1 2,4-D and 0.5 mg·L-1 BAP, pH5.8. The protoplast suspension was transferred to a 24-well plate and incubated for 48 hours at 28° C. in dark.

For each treatment, one aliquot was collected and analyzed cytometrically in a BD Accuri C6 flow cytometer to determine the proportion of fluorescent cells and an aliquot was collected for Illumina sequencing library preparation as follows.

Protoplasts were harvested by centrifugation for 10 minutes at 1000 rpm. The supernatant was discarded and the pellet frozen in liquid nitrogen. DNA was extracted using the Qiagen Plant DNeasy kit.

A primary amplicon spanning the mutation site was generated using the following PCR primers:

(SEQ ID NO: 22)
5′-GAGCTGAGGCTAGGCATCATC-3′;
and
(SEQ ID NO: 23)
5′-TCCATCCTCGATGTTGTGCC-3′.

The resulting amplicon was subsequently used for the preparation of an Illumina sequencing library and sequenced on a MiSeq sequencer.

Sequence data analysis was performed using a dedicated analysis script in Galaxy and formatted in MS Excel using a dedicated script in VBA.

InDel frequencies were determined as the proportion of reads containing InDels at the expected Cas9 cutting site over the total number of reads for the sample under consideration.

Similarly, gene targeting frequencies were determined as the number of reads containing the expected causative mutation (the mutation responsible for the restoration of YFP functionality) over the total number of reads for the sample under consideration.

### 4. Results

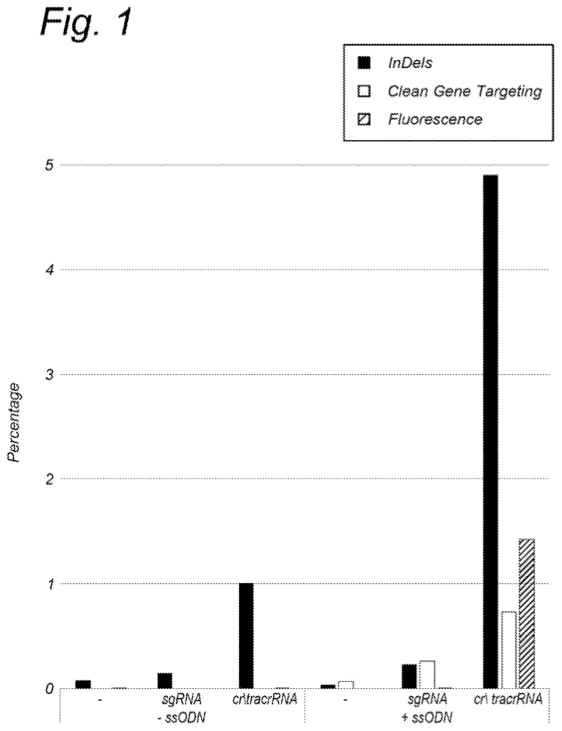

The results of the Experiment are summarized in FIG. 1. It appears clearly that the dgRNA (comprising crRNA and tracrRNA as individual molecules) outperformed the sgRNA at least 6 to 7 fold, both for inducing indels as well as for clean gene targeting, i.e. inducing the SNP restoring the GFP open reading frame. Results from the in vitro sgRNA and synthetic sgRNA were similar (data not shown).

Sequence of the open reading frame of DsRED::YFP stop fluorescent reporter construct with underscored: 6×HIS-NLS; normal font: DsRED; italics: YFP with in bold the point mutation resulting in a stop codon (SEQ ID NO: 15):

ATGGGAAGAGGATCGCATCACCACCATCATCATAAGCTTCCAAAGAAGAA
GAGGAAGGTTCTCGAGACCATGATCACTCCATCTCTTCATGCTTGCAGGT
CTACTCTTGAGGATCCAAGAGTTCCAGTGGCTACTATGGATTCTACTGAG
AACGTGATCAAGCCATTCATGAGGTTCAAGGTTCACATGGAAGGATCTGT
TAACGGACACGAGTTTGAAATTGAAGGTGAAGGTGAGGGAAAGCCATATG
AAGGTACTCAAACTGCTAAGCTTCAGGTTACAAAGGGTGGACCACTTCCA
TTTGCTTGGGATATTTTGTCTCCACAGTTCCAGTACGGATCTAAGGTTTA
CGTGAAACACCCAGCTGATATCCCAGATTACAAGAAGTTGTCTTTCCCAG
AGGGATTCAAGTGGGAGAGGGTTATGAATTTTGAGGATGGTGGTGTGGTG
ACTGTGACTCAAGATTCTTCACTTCAGGATGGAACTTTCATCTACCACGT
GAAGTTCATCGGAGTGAACTTTCCATCTGATGGACCAGTGATGCAGAAAA
AAACTCTTGGATGGGAGCCATCTACTGAGAGACTTTATCCAAGGGATGGT
GTTCTTAAGGGTGAGATTCACAAGGCTCTTAAGCTTAAAGGTGGTGGACA
CTACCTTGTTGAGTTCAAGTCTATCTACATGGCTAAGAAGCCAGTTAAGC
TTCCTGGTTACTACTATGTGGATTCTAAGCTTGATATCACTTCTCACAAC
GAGGATTACACTGTGGTTGAGCAATATGAGAGAGCTGAGGCTAGGCATCA
TCTTTTTCAGTACCTTGAGATGGTGTCAAAGGGTGAAGAGTTGTTCACTG
GTGTGGTTCCAATCCTTGTTGAGCTTGATGGTGATGTGAACGGACATAAG
TTCTCTGTTTCTGGTGAGGGTGAAGGTGATGCTACTTAAGGTAAACTTAC
ACTTAAGTTCATCTGCACTACTGGAAAGCTTCCAGTTCCATGGCCAACTC
TTGTTACTACTTTCGGATACGGTGTTCAATGCTTCGCTAGGTATCCAGAT
CATATGAGGCAGCACGATTTCTTCAAGTCTGCTATGCCAGAGGGATATGT
TCAAGAGAGGACTATCTTCTTCAAGGATGATGGAAACTACAAGACTAGGG
CTGAGGTTAAGTTCGAGGGTGATACTCTTGTGAACAGGATTGAGCTTAAG
GGAATCGATTTCAAAGAGGATGGAAACATCCTTGGACACAAGCTTGAGTA
CAACTACAACTCTCACAACGTGTACATCATGGCTGATAAGCAGAAGAACG
GAATCAAGGTTAACTTCAAGATCAGGCACAACATCGAGGATGGATCTGTT
CAACTTGCTGATCATTACCAGCAGAACACTCCAATTGGAGATGGACCAGT
TCTTTTGCCAGATAACCACTACCTTTCTTACCAGTCTGCTCTTTCTAAGG
ATCCAAACGAGAAGAGGGATCACATGGTTCTTTTGGAGTTCGTTACTGCT
GCTGGAATCACTCTTGGAATGGATGAGCTTTACAAGTGAG

1. A method for targeted modification of DNA in a plant cell, comprising a step of contacting the DNA with an RNA-guided CRISPR-system nuclease complex, wherein said complex comprises a CRISPR-system nuclease, a crRNA and a tracrRNA and wherein the crRNA and the tracrRNA are separate (non-covalently linked) molecules.

2. The method according to claim 1, wherein the CRISPR-system nuclease comprises two catalytically active endonuclease domains.

3. The method according to claim 1, wherein the CRISPR-system nuclease comprises at least one catalytically inactive endonuclease domain.

4. The method according to claim 1, wherein the CRISPR-system nuclease is fused to a functional domain, preferably, a deaminase domain.

5. The method according to claim 1, wherein the CRISPR-system nuclease is introduced in the cell by transfecting the cell with a vector encoding said CRISPR-system nuclease.

6. The method according to claim 1, wherein the CRISPR-system nuclease is introduced in the cell by transfecting the cell with the CRISPR-system nuclease.

7. The method according to claim 1, wherein at least one of the crRNA and tracrRNA is introduced in the cell by transfecting the cell with a vector encoding said crRNA and/or tracrRNA.

8. The method according to claim 1, wherein at least one of the crRNA and tracrRNA is introduced in the cell by transfecting the cell with said crRNA and/or tracrRNA, and wherein preferably the crRNA and/or tracrRNA is chemically modified.

9. The method according to claim 1, wherein the cell is further transfected with a template oligonucleotide, wherein preferably the template oligonucleotide is chemically modified.

10. The method according to claim 1, wherein the cell is further transfected with a donor construct, wherein preferably the donor construct is chemically modified.

11. The method according to claim 1, wherein the CRISPR-system endonuclease, crRNA, tracrRNA and/or optionally the template oligonucleotide or donor construct, are introduced into the plant cell using polyethylene glycol mediated transfection, preferably using an aqueous medium comprising PEG.

12. The method according to claim 1, wherein the method further comprises the step of regenerating a plant or descendant thereof comprising the targeted modification.

13. An RNA-guided CRISPR-system nuclease complex comprising the CRISPR-system nuclease, the crRNA and the tracrRNA as defined in claim 1, or one or more constructs encoding the same, for targeted modification of DNA in a plant cell.

14. A kit for targeted modification of DNA in a plant cell comprising at least one of
i) a container comprising the CRISPR-system nuclease of claim 13; and
ii) a container comprising one or more constructs of claim 13,
and optionally a container comprising a tracrRNA and/or one or more crRNAs, and/or constructs encoding the same.

15. Use of a RNA guided CRISPR-system nuclease complex as defined in claim 13, or one or more constructs encoding the same, or a kit as defined in claim 14, for targeted modification of DNA in a plant cell.


SEQUENCE LISTING (25 sequences)

SEQ ID NO: 1 - DNA, 8 bp, artificial sequence
  PAM
  misc_feature (1)..(4): n is a, c, g, or t
  misc_feature (6)..(7): n is a, c, g, or t
nnnncnna

SEQ ID NO: 2 - DNA, 684 bp, Artificial Sequence
  rAPOBEC1 encoding sequence
atgagcagcgaaaccggtccggttgcagttgatccgaccctgcgtcgtcgtattgaaccgcatgaatttgaagttttttttgatccgcgtgaactgcgtaaagaaacctgtctgctgtatgaaattaattggggtggtcgtcatagcatttggcgtcataccagccagaataccaataaacatgttgaagttaattttattgaaaaatttaccaccgaacgttatttttgtccgaatacccgttgtagcattacctggtttctgagctggagcccgtgtggtgaatgtagccgtgcaattaccgaatttctgagccgttatccgcatgttaccctgtttatttatattgcacgtctgtatcatcatgcagatccgcgtaatcgtcagggtctgcgtgatctgattagcagcggtgttaccattcagattatgaccgaacaggaaagcggttattgttggcgtaattttgttaattatagcccgagcaatgaagcacattggccgcgttatccgcatctgtgggttcgtctgtatgttctggaactgtattgtattattctgggtctgccgccgtgtctgaatattctgcgtcgtaaacagccgcagctgaccttttttaccattgcactgcagagctgtcattatcagcgtctgccgccgcatattctgtgggcaaccggtctg

SEQ ID NO: 3 - DNA, 681 bp, Artificial Sequence
  rAPOBEC1 encoding sequence
atgagcagcgaaacaggtccggttgcagttgatccgaccctgcgtcgtcgtattgaaccgcatgaatttgaagttttttttgatccgcgtgagctgcgtaaagaaacctgtctgctgtatgaaattaactggggtggtcgtcatagcatttggcgtcataccagccagaataccaataaacatgtggaagtgaacttcatcgagaaatttaccaccgaacgttatttttgtccgaatacccgttgtagcattacctggtttctgagctggtcaccgtgtggtgaatgtagccgtgcaattaccgaatttctgagccgttatccgcatgttaccctgtttatctatattgcccgtctgtatcatcatgcagatccgcgtaatcgtcagggtctgcgtgatctgattagcagcggtgttaccattcagattatgaccgaacaagaaagcggttattgctggcgtaattttgtgaattatagcccgagcaatgaagcacattggcctcgctatccgcatctgtgggttcgtctgtatgttctggaactgtattgtattattctgggtctgcctccgtgtctgaatattctgcgtcgtaaacagccgcagctgaccttttttaccattgcactgcagagctgtcattatcagcgtctgccaccgcatattctgtgggcaacaggt

SEQ ID NO: 4 - protein, 228 aa, Artificial Sequence
  rAPOBEC1
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGL

SEQ ID NO: 5 - DNA, 501 bp, Artificial Sequence
  TadA encoding sequence
atgtccgaggtcgagttctctcatgagtactggatgaggcacgctctcactcttgctaaaagagcttgggacgagagagaggttccagttggagctgttttggtgcacaacaaccgtgtgattggcgaaggatggaacaggccaattggaaggcatgatccaactgctcacgctgagattatggcccttagacaaggtggactcgtgatgcagaactacaggcttatcgacgccactctctacgtgacacttgagccatgtgttatgtgcgctggtgccatgattcactccaggattggaagggttgtgttcggagctagagatgctaaaactggcgctgccggatctctcatggatgtgcttcatcatcctgggatgaaccacagggttgagatcactgagggaatccttgctgatgagtgcgctgctctcctgtctgattttttcaggatgaggcgtcaagagatcaaggcccagaagaaggctcagtcctctactgat

SEQ ID NO: 6 - protein, 167 aa, Artificial Sequence
  TadA
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 7 - protein, 5 aa, Artificial Sequence
  linker
EAAAK

SEQ ID NO: 8 - protein, 11 aa, Artificial Sequence
  linker
SPKKKRKVEAS

SEQ ID NO: 9 - protein, 16 aa, artificial sequence
  linker
SGSETPGTSESATPES

SEQ ID NO: 10 - protein, 17 aa, Artificial sequence
  linker
KSGSETPGTSESATPES

SEQ ID NO: 11 - DNA, 252 bp, Artificial Sequence
  UGI encoding sequence
atgactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctc

SEQ ID NO: 12 - DNA, 168 bp, Artificial Sequence
  UGI encoding sequence
accaatctgagcgatatcattgaaaaagaaaccggcaaacagctggtgattcaagaaagcattctgatgctgcctgaagaagtggaagaagttattggtaataaaccggaaagcgatattctggttcataccgcatatgatgaaagcaccgatgaaaatgttatgctg

SEQ ID NO: 13 - protein, 84 aa, Artificial Sequence
  UGI
MTNLSDIIEKETGKQLVIQESILMLPEEVEEVIGNKPESDILVHTAYDESTDENVMLLTSDAPEYKPWALVIQDSNGENKIKML

SEQ ID NO: 14 - protein, 56 aa, Artificial sequence
  UGI
TNLSDIIEKETGKQLVIQESILMLPEEVEEVIGNKPESDILVHTAYDESTDENVML

SEQ ID NO: 15 - DNA, 1540 bp, artificial sequence
  artificial sequence
atgggaagaggatcgcatcaccaccatcatcataagcttccaaagaagaagaggaaggttctcgagaccatgatcactccatctcttcatgcttgcaggtctactcttgaggatccaagagttccagtggctactatggattctactgagaacgtgatcaagccattcatgaggttcaaggttcacatggaaggatctgttaacggacacgagtttgaaattgaaggtgaaggtgagggaaagccatatgaaggtactcaaactgctaagcttcaggttacaaagggtggaccacttccatttgcttgggatattttgtctccacagttccagtacggatctaaggtttacgtgaaacacccagctgatatcccagattacaagaagttgtctttcccagagggattcaagtgggagagggttatgaattttgaggatggtggtgtggtgactgtgactcaagattcttcacttcaggatggaactttcatctaccacgtgaagttcatcggagtgaactttccatctgatggaccagtgatgcagaaaaaaactcttggatgggagccatctactgagagactttatccaagggatggtgttcttaagggtgagattcacaaggctcttaagcttaaaggtggtggacactaccttgttgagttcaagtctatctacatggctaagaagccagttaagcttcctggttactactatgtggattctaagcttgatatcacttctcacaacgaggattacactgtggttgagcaatatgagagagctgaggctaggcatcatctttttcagtaccttgagatggtgtcaaagggtgaagagttgttcactggtgtggttccaatccttgttgagcttgatggtgatgtgaacggacataagttctctgtttctggtgagggtgaaggtgatgctacttaaggtaaacttacacttaagttcatctgcactactggaaagcttccagttccatggccaactcttgttactactttcggatacggtgttcaatgcttcgctaggtatccagatcatatgaggcagcacgatttcttcaagtctgctatgccagagggatatgttcaagagaggactatcttcttcaaggatgatggaaactacaagactagggctgaggttaagttcgagggtgatactcttgtgaacaggattgagcttaagggaatcgatttcaaagaggatggaaacatccttggacacaagcttgagtacaactacaactctcacaacgtgtacatcatggctgataagcagaagaacggaatcaaggttaacttcaagatcaggcacaacatcgaggatggatctgttcaacttgctgatcattaccagcagaacactccaattggagatggaccagttcttttgccagataaccactacctttcttaccagtctgctctttctaaggatccaaacgagaagagggatcacatggttcttttggagttcgttactgctgctggaatcactcttggaatggatgagctttacaagtgag

SEQ ID NO: 16 - DNA, 20 bp, artificial sequence
  artificial sequence
tctgtttctggtgagggtga

SEQ ID NO: 17 - RNA, 22 nt, artificial sequence
  crRNA linker
guuuuagagcuguguuguuucg

SEQ ID NO: 18 - RNA, 73 nt, artificial sequence
  tracrRNA
cgaaacagcauagcaaguuaaaauaaggcuaguccguuaucaacuugaaaaaguggcaccgagucggugcuuu

SEQ ID NO: 19 - RNA, 80 nt, artificial sequence
  sgRNA scaffold
guuuuagagcuagaaauagcaaguuaaaauaaggcuaguccguuaucaacuugaaaaaguggcaccgagucggugcuuuu

SEQ ID NO: 20 - RNA, 102 nt, artificial sequence
  sgRNA
gucuguuucuggugagggugacguuuuagagcuagaaauagcaaguuaaaauaaggcuaguccguuaucaacuugaaaaaguggcaccgagucggugcuuuu

SEQ ID NO: 21 - DNA, 127 bp, artificial sequence
  ssODN
  phosphorothioate linkage (1)..(2)
  phosphorothioate linkage (126)..(127)
agtagtaacaagagttggccatggaactggaagctttccagtagtgcagatgaacttaagtgtaagtttaccgtaagtagcatcaccttctccttctcctgaaacagagaacttatgtccgttcaca

SEQ ID NO: 22 - DNA, 21 bp, artificial sequence
  PCR primer
gagctgaggctaggcatcatc

SEQ ID NO: 23 - DNA, 20 bp, artificial sequence
  PCR primer
tccatcctcgatgttgtgcc

SEQ ID NO: 24 - RNA, 86 nt, artificial sequence
  tracerRNA
ggaaccauucaaaacagcauagcaaguuaaaauaaggcuaguccguuaucaacuugaaaaaguggcaccgagucggugcuuuuuuu

SEQ ID NO: 25 - RNA, 22 nt, artificial sequence
  linker crRNA
guuuuagagcuaugcuguuuug